(19) United States
(12) Patent Application Publication
(10) Pub. No.: US 2017/0239332 A1
(43) Pub. Date: Aug. 24, 2017

(54) Regeneration of Inner Ear Cells

(71) Applicant: WAKE FOREST UNIVERSITY HEALTH SCIENCES, Winston Salem, NC (US)

(72) Inventors: Joseph Charles BURNS, Chapel Hill, NC (US); John D. JACKSON, Clemmons, NC (US)

(21) Appl. No.: 15/371,966

(22) Filed: Dec. 7, 2016

Related U.S. Application Data
(63) Continuation of application No. 14/382,656, filed on Sep. 3, 2014, now abandoned.
(60) Provisional application No. 61/606,675, filed on Mar. 5, 2012.

(51) Int. Cl. A61K 38/17; C12N 5/071; A61K 45/06; A61K 48/00; C12N 7/00

(52) U.S. Cl. CPC A61K 38/1761; A61K 48/005; C12N 7/00; A61K 45/06; C12N 5/0627; C12N 2710/10043; C12N 2710/10032; C12N 2710/10071; C12N 2506/09; C12N 2510/00

(57) ABSTRACT

The invention provides compositions and methods for treating or preventing hearing loss in a subject. The method comprises administering to the subject in need thereof, at least Myc or an agent that increases the expression of Myc in an inner ear organ, or associated neural structures, of the subject so as to treat or prevent the hearing loss.

### CROSS-REFERENCE TO RELATED APPLICATION

The present application is a continuation of, and claims priority to, U.S. patent application Ser. No. 14/382,656, filed Sep. 3, 2014, which is a 35 U.S.C. §371 national phase application of, and claims priority to, PCT Application No. PCT/US2013/027985, filed Feb. 27, 2013, which claims priority under 35 U.S.C. §119(e) to U.S. Provisional Application Ser. No. 61/606,675, filed Mar. 5, 2012, all of which applications are incorporated herein by reference in their entireties.

### BACKGROUND OF THE INVENTION

The sensory epithelia within the inner ears of adult mammals and humans are highly differentiated, postmitotic, and regeneration deficient. Thus, the loss of sound- and acceleration-detecting hair cells from auditory or vestibular sensory epithelia leads to permanent hearing or balance impairments, respectively. In contrast, the less differentiated sensory epithelia within the inner ears of developing mice and non-mammals of all ages are capable of more significant hair cell regeneration after damage, and non-mammals can recover sensory function (Corwin and Cotanche, 1988, Science 240:1772-1774; Ryals and Rubel, 1988, Science 240:1774-1776; Warchol, 2011, Hearing Research 273:72-79; Burns et al., 2012, J. Neurosci. 32:6570-6577; Kelley et al., 1995, J. Neurosci. 15:3013-3026).

During sensory epithelial development and regeneration, cells that morphologically resemble supporting cells act as otic progenitors that can self-renew and give rise to new hair cells. In vitro and in vivo evidence suggests that the progressive, postnatal depletion of these progenitors, likely via terminal differentiation, limits regeneration in mammals (Burns et al., 2012, J. Neurosci. 32:6570-6577; Li et al., 2003, Nat. Med. 9:1293-1299; Oshima et al., 2007, J. Assoc. Res. Otolaryngol. 8:18-31; White et al., 2006, Nature 441:984-987; Zhai et al., 2005, Journal of Neurobiology 65:282-293; Collado et al., 2009, PloS One 6:e23861; Davies et al., 2007, Eur. J. Neurosci. 25:985-998; Gu et al., 2007, Eur. J. Neurosci. 25:1363-1372; Lu and Corwin, 2008, Dev. Neurobiol. 68:1059-1075; Hume et al., 2003, J. Assoc. Res. Otolaryngol. 4:422-443; Meyers and Corwin, 2007, J. Neurosci. 27:4313-4325; Zheng et al., 1997, J. Neurosci. 17:216-226; Ruben, 1967, Acta Otolaryngol. (Suppl.) 220:1-44; Burns et al., 2012, J Assoc. Res. Otalaryngol. 13(5):609-27; Burns et al., 2008, J. Comp. Neurol. 511:396-414).

Ectopic, long-term expression of the four transcription factors, Oct3/4, Sox2, Klf4, and c-Myc reprograms isolated somatic cells into induced pluripotent stem cells (iPSCs) (Takahashi et al., 2007, Cell 131:861-872; Ho et al., 2011, J. Cell. Physiol. 226:868-878; Yu et al., 2007, Science 318:1917-1920; Takahashi and Yamanaka, 2006, Cell 126:663-676). The initial stages of the reprogramming process result in a partially dedifferentiated, “pre-iPSC” state, and transient expression of the iPSC factors has recently been utilized to directly reprogram somatic cells into lineage-restricted, multipotent progenitor/stem cells (Kim et al., 2011, Proc. Natl. Acad. Sci. U S A. 108:7838-7843; Efe et al., 2011, Nat. Cell. Biol. 13:215-222; Meissner et al., 2007, Nat. Biotechnol. 25:1177-1181; Sridharan et al., 2009, Cell 136:364-377; Silva et al., 2008, PLoS Biol 6:e253; Mikkelsen et al., 2008, Nature 454:49-55).

Thus, there is a need in the art for a therapy that can induce robust proliferation and replacement of inner ear cells to restore hearing and vestibular (i.e. detection of gravity and angular acceleration) function in humans that have significant sensory deficits resulting from the damage or loss of those cells. The present invention satisfies this need.

### SUMMARY OF THE INVENTION

The invention provides a method for stimulating the formation of an inner ear sensory hair cell from an inner ear supporting cell. In one embodiment, the method comprises contacting an inner ear supporting cell with an effective amount of an agent that is capable of inducing the expression of a Myc family protein in the supporting cell, wherein expression of the Myc family protein in the inner ear supporting cell can stimulate the formation of an inner ear sensory hair cell.

In one embodiment, the Myc family protein is selected from the group consisting of c-Myc, N-Myc, L-Myc, and any combination thereof.

In one embodiment, the agent is an expression vector comprising a nucleic acid sequence encoding a Myc family protein.

In one embodiment, the vector is an adenovirus vector or an AAV.

In one embodiment, wherein after a period of time where the Myc family protein is expressed in the inner ear supporting cell, the expression of the Myc family protein is inhibited by contacting the supporting cell with an inhibitor.

In one embodiment, the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

The invention provides a method of treating or preventing hearing loss in a mammal. In one embodiment, the method comprises administering to the mammal in need thereof, an effective amount of an agent that is capable of inducing the expression of a Myc family protein in an inner ear supporting cell of the mammal, wherein expression of the Myc family protein in the inner supporting cell can stimulate the formation of an inner ear sensory hair cell.

The invention provides a method for stimulating the formation of an inner ear sensory hair cell from an inner ear supporting cell. In one embodiment, the method comprises contacting an inner ear supporting cell with an effective amount of an agent that is capable of inducing the expression of cyclin D1 in the supporting cell, wherein expression of cyclin D1 in the inner ear supporting cell can stimulate the formation of an inner ear sensory hair cell.

The invention provides a method of treating or preventing hearing loss in a mammal. In one embodiment, the method comprises administering to the mammal in need thereof, an effective amount of an agent that is capable of inducing the expression of cyclin D1 in an inner ear supporting cell of the mammal, wherein expression of cyclin D1 in the inner supporting cell can stimulate the formation of an inner ear sensory hair cell.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1L, depict how adenovirus primarily infects supporting cells but also some hair cells in adult mouse utricles in vitro. FIGS. 1A-1D depict low magnification (20×/0.75 NA) confocal images of utricles from adult mice that were incubated with increasing concentrations of adenovirus engineered to express green fluorescent protein (GFP) under the control of a CMV promoter. Utricles were fixed and labeled 3 d after adenovirus was washed out. Hair cells are labeled with antibodies to myosin VIIA. FIG. 1E is an image depicting a high-resolution (63×/1.4 NA) confocal section taken at the apical surface of the sensory epithelium shows a cluster of GFP-positive supporting cells. The supporting cell apical surfaces have characteristic polygonal shapes compared to the circular profile of hair cells labeled with anti-myosin-VIIA. FIG. 1F is an image depicting a view of a confocal image stack parallel to the apical-basal axis of supporting cells and hair cells shows a supporting cell expressing GFP, but its neighboring myosin-VIIA-labeled hair cell does not. Scale bar, 5 μm. FIG. 1G is an image depicting the same view as in FIG. 1F showing a GFP-expressing hair cell. Scale bar, 5μm. FIG. 1H is a graph depicting the quantification of the percentage of GFP-expressing supporting cells (black circles) and hair cells with increasing concentration of adenovirus. Myosin VIIA labeling was used to distinguish between supporting cells and hair cells, and sigmoidal equations were fit to the data points (see Table 1 for equation and coefficients). FIGS. 1I-1L are images depicting fluorescent phalloidin labeling in the utricles from 1A-1D. Scale bar=100 μm.

FIGS. 2A-2FF, depict how co-infection of Ad.O, Ad.K, Ad. S, and Ad.MT58A induces c ell cycle reentry of postmitotic supporting cells in adult mouse utricles in vitro. FIG. 2A is an images depicting a low magnification (20×/0.75 NA) confocal image of co-infected utricles from adult mice at 1 day post-virus (DPV). FIG. 2B is an image depicting a low magnification (20×/0.75 NA) confocal image of co-infected utricles from adult mice at 3 DPV. FIG. 2C is an image depicting a low magnification (20×/0.75 NA) confocal image of co-infected utricles from adult mice at 5 DPV. FIG. 2D is an image depicting a low magnification (20×/0.75 NA) confocal image of co-infected utricles from adult mice at 7 DPV. FIG. 2E is an image depicting a low magnification (20×/0.75 NA) confocal image of co-infected utricles from adult mice at 10 DPV. FIG. 2F is an image depicting a low magnification (20×/0.75 NA) confocal image of co-infected utricles from adult mice at 14 DPV. FIG. 2G is an image depicting a low magnification (20×/0.75 NA) confocal image of co-infected utricles from adult mice at 21 DPV. FIG. 2H is an image depicting a low magnification (20×/0.75 NA) confocal image of co-infected utricles from adult mice at 28 DPV. BrdU was included in the culture medium for the entire period after virus washout, and utricles were fixed and labeled at the times indicated. Hair cells and nuclei that entered S-phase are labeled with antibodies to myosin VIIA and BrdU, respectively. FIG. 21 is an image depicting a low magnification (20×/0.75 NA) confocal image of control utricles infected with Ad.GFP at 1 DPV. FIG. 2J is an image depicting a low magnification (20×/0.75 NA) confocal image of control utricles infected with Ad.GFP at 3 DPV. FIG. 2K is an image depicting a low magnification (20×/0.75 NA) confocal image of control utricles infected with Ad.GFP at 5 DPV. FIG. 2L is an image depicting a low magnification (20×/0.75 NA) confocal image of control utricles infected with Ad.GFP at 7 DPV. FIG. 2M is an image depicting a low magnification (20×/0.75 NA) confocal image of control utricles infected with Ad.GFP at 10 DPV. FIG. 2N is an image depicting a low magnification (20×/0.75 NA) confocal image of control utricles infected with Ad.GFP at 14 DPV. FIG. 2O is an image depicting a low magnification (20×/0.75 NA) confocal image of control utricles infected with Ad.GFP at 21 DPV. FIG. 2P is an image depicting a low magnification (20×/0.75 NA) confocal image of control utricles infected with Ad.GFP at 28 DPV. Few to no BrdU labeled nuclei are present in the sensory epithelium. FIGS. 2Q-2X are images depicting fluorescent phalloidin labeling in the utricles from FIGS. 2A-2H, respectively. Scale bar=100 μm. FIGS. 2Y-2FF are images depicting GFP expression in the utricles from FIGS. 2I-2P, respectively. Scale bar=100 μm.

FIGS. 3A-3E, depict how BrdU-labeled cells in utricles co-infected with Ad.O, Ad.K, Ad. S, and Ad.MT58A are present after four weeks in culture and many appear in doublets. FIG. 3A is an image depicting a high-resolution (63×/1.4 NA) view orthogonal to the apical-basal axis of supporting cells and hair cells in a co-infected utricle that was cultured for 7 days post-virus. FIG. 3B is an image depicting a high-resolution (63×/1.4 NA) view parallel to the apical-basal axis of supporting cells and hair cells in a co-infected utricle that was cultured for 7 days post-virus. BrdU was included in the culture medium for the entire period after virus washout. Hair cells and nuclei that have entered S-phase are labeled with antibodies to myosin VIIA and BrdU, respectively. The two BrdU-positive nuclei in B appear to be a division pair, with one positioned at the level of hair cell nuclei and the other at the supporting cell nuclear layer. Scale bar in A, 20 μm. Scale bar in B, 5 μm. FIG. 3C is a graph depicting quantification of the mean number of BrdU-positive nuclei per sensory epithelium versus time in culture. Data from co-infected utricles are shown in gray, and data from control utricles infected with GFP are shown in black. FIG. 3D is a graph depicting the percentage of BrdU-positive nuclei that appeared as doublets in co-infected utricles (same utricles used for the gray data points in FIG. 3C). Subtracting the percentage from 100 yields the percentage of nuclei that appeared as singlets. FIG. 3E is a confocal image of a co-infected utricle fixed at 8 days post-virus and labeled with antibodies to BrdU and activated caspase 3. Arrow points to a pyknotic nucleus that labeled with both antibodies. Scale bar, 10 μm.

FIGS. 4A-4H, depict how infection with Ad.O, Ad.K, or Ad.MT58A leads to detectable increases in Oct3/4, Klf4, or c-Myc protein levels in some supporting cells. FIG. 4A is an image of utricles infected with Ad.O and labeled with Oct 3/4. FIG. 4B is an image of utricles infected with Ad.GFP and labeled with Oct 3/4. FIG. 4C is an image of utricles infected with Ad.K and labeled with Klf4. FIG. 4D is an image of utricles infected with Ad.GFP and labeled with Klf4. FIG. 4E is an image of utricles infected with Ad.S and labeled with Sox2. FIG. 4F is an image of utricles infected with Ad.GFP and labeled with Sox2. FIG. 4G is an image of utricles infected with Ad.MT58A and labeled with c-Myc. FIG. 4H is an image of utricles infected with Ad.GFP and labeled with c-Myc. Utricles were infected with 5×108 TU/mL of virus and fixed at 3 days post-virus. Shown are single confocal slices (0.75 μm z-thickness) at the level of the supporting cell nuclei. Supporting cell nuclei are labeled with the DNA dye, DRAQS (blue). The same amplifier gain and offset settings used to acquire images of the treated samples were used for acquiring images of their respective Ad.GFP controls. Note the similar intensity of Sox2 labeling in utricles infected with Ad.S and Ad.GFP. Scale bar=10 μm.

FIGS. 5A-5J, depict how infection with Ad.MT58A is both necessary and sufficient for the observed proliferative response. FIG. 5A is a confocal images of utricles that were individually infected with Ad.O. FIG. 5B is a confocal images of utricles that were individually infected with Ad.K. FIG. 5C is a confocal images of utricles that were individually infected with Ad.S. FIG. 5D is a confocal images of utricles that were individually infected with Ad.MT58A. Utricles were cultured in the presence of BrdU for 5 days post-virus (adenovirus concentrations equal to those used for co-infection experiments). Significant BrdU labeling is only detected in the sensory epithelium of a utricle infected with Ad.MT58A. Hair cells are labeled with an antibody to myosin VIIA. FIG. 5E is a confocal images of utricles infected with 1×109 TU/mL (5×) of Ad.MT58A. FIG. 5F is a confocal images of utricles infected with 2×109 TU/mL (10×) of Ad.MT58A. Inset in FIG. 5F shows a zoomed region of the sensory epithelium. Scale bar for inset, 20 μm. FIG. 5J is a graph depicting quantification of the number of BrdU-labeled nuclei per sensory epithelium for the different adenovirus combinations tested. O: Ad.O, K: Ad.K, S: Ad.S, M: Ad.MT58A, OKSM =co-infection with Ad.O, Ad.K, Ad.S, and Ad.M T58A. The difference in the number of BrdU-positive nuclei in co-infected utricles and utricles infected with Ad.MT58A did not reach statistical significance, but the increases in BrdU-positive nuclei at higher concentrations of Ad.MT58A were significant (asterisks indicate a significant difference from all other conditions; p<0.05; One-way ANOVA with Tukey's Test of Multiple Comparisons; n=4 utricles). FIGS. 5G-5I are confocal images of the BrdU channel in FIGS. 5D-5F, respectively, without the myosin VIIA overlay. White dashed lines demarcate the borders of the sensory epithelium. Scale bar=100 μm.

FIGS. 6A-6F, depict how supporting cells that reenter the cell cycle after Ad.MT58A infection can progress to mitosis. FIG. 6A is a series of confocal images depicting an Ad.MT58A-infected utricle (1×109 TU/mL) that was fixed at 7 days post-virus (DPV) and co-labeled with antibodies to BrdU and Ki-67. Phalloidin labeling (grayscale) is shown to aid in visualizing the borders of the sensory epithelium. White dashed lines demarcate the borders of the sensory epithelium. Scale bar=100 μm. Insets show high-resolution views of nuclei in the sensory epithelium. Arrows indicate nuclei that labeled with antibodies to BrdU but not Ki67. Scale bar for insets, 5μm. FIG. 6B is a graph depicting the mean number of BrdU-labeled nuclei and Ki-67-labeled nuclei per sensory epithelium at 5, 7, and 10 DPV. FIG. 6C is a graph depicting quantification of the percentage of the BrdU-positive population that did not label with Ki67 antibodies and the percentage of the Ki-67-positive population that did not label with BrdU antibodies. FIG. 6D is a confocal image of an adult mouse utricle infected with Ad.MT58A (1×109 TU/mL) that was fixed at 7 DPV and co-labeled with antibodies to PH3-Ser10 (white) and Ki-67. FIG. 6E is a series of confocal images of a utricle from an embryonic day 17.5 (E17.5) mouse that was fixed in vivo and co-labeled with antibodies to PH3-Ser10 (white) and Ki-67. Phalloidin labeling (grayscale) is shown to aid in visualizing the borders of the sensory epithelium. White dashed lines demarcate the borders of the sensory epithelium. Arrows in FIGS. 6D and 6E indicate PH3-Ser10/Ki-67 co-labeled nuclei. Scale bar for FIGS. 6D and 6E=100 μm. FIG. 6F is a graph depicting quantification of the percentage of the Ki-67-positive populations that labeled with antibodies to PH3-Ser10. The difference in the percentage of PH3-Ser10-positive/Ki-67-positive nuclei did not reach statistical significance (p>0.05; Student's t-test). The numbers above the gray bars indicate the mean number of PH3-Ser10-labeled nuclei per sensory epithelium. There were significantly more PH3-Ser10-positive nuclei in E17.5 utricles (p<0.05; Student's t-test).

FIGS. 7A-7H, depict how some supporting cells in Ad.MT58A-infected utricles survive for weeks in culture after reentering the cell cycle. FIG. 7A is diagram depicting the BrdU labeling paradigm. BrdU was either included in the culture medium for the entire culture period after infection with adenovirus, or it was washed out at 5 days post-virus (DPV) and utricles were cultured for an additional 5 d in its absence. FIG. 7B is a confocal image depicting Ad.MT58A-infected utricles (1×109 TU/mL) fixed at 10 DPV after being cultured with BrdU from 1-10 DPV. FIG. 7C is a confocal image depicting Ad.MT58A-infected utricles (1×109 TU/mL) fixed at 10 DPV after being cultured with BrdU from 1-5 DPV. Scale bar for FIGS. 7B-7C=100 μm. FIG. 7D is a graph depicting quantification of the number of BrdU-labeled cells in the sensory epithelium from utricles cultured as depicted in A (gray and white bars). Quantification of 5 DPV BrdU labeling (same as depicted in FIG. 5J) is shown to visualize the decline in BrdU-labeled cells from 5 DPV (black bars) to 10 DPV. Data shown is from co-infection experiments (OKSM), infection with 2×108 TU/mL Ad.MT58A (1×M), and infection with 1×109 TU/mL Ad.MT58A (5×M). FIG. 7E is a confocal image of an Ad.MT58A-infected utricle (1×109 TU/mL) fixed at 21 DPV after being cultured with BrdU from 1-5 DPV. Antibody labeling for BrdU and Ki-67 is shown. Scale bar=100 μm. FIG. 7F is a confocal image of an Ad.MT58A-infected utricle (1×109 TU/mL) fixed at 21 DPV after being cultured with BrdU from 18-21 DPV. Antibody labeling for BrdU and myosin VIIA is shown. Scale bar=100 μm. FIG. 7G is a confocal image of an Ad.MT58A-infected utricle fixed at 10 DPV after switching from growth medium to differentiation medium at 5 DPV. BrdU was included in the medium throughout. Scale bar=100 μm. FIG. 7H is a graph depicting the mean number of BrdU-positive nuclei per sensory epithelium at 5, 10, and 14 DPV for the experiment described in FIG. 7G. White dashed lines demarcate the borders of the sensory epithelium in all panels.

FIGS. 8A-8E, depict how supporting cells that reenter the cell cycle in Ad.MT58A-infected utricles may be capable of differentiating towards a hair-cell-like fate. Shown are confocal images taken from Ad.MT58A-infected utricles (1×109 TU/mL) fixed at 10 days post-virus (DPV) and labeled with antibodies to BrdU and myosin VIIA. Growth medium was exchanged for differentiation medium at 5 DPV. FIGS. 8A and 8B are confocal images depicting views parallel to the long axis of the hair cells. The arrowhead points to a BrdU-positive/myosin-VIIA-positive cell that does not extend to the apical surface and is probably damaged or dying. The other BrdU-positive/myosin-VIIA-positive cells extend from the hair nuclear layer up to the apical surface, where they appear to display tiny, bundle-like projections. BrdU-positive/myosin-VIIA-positive cells are located within close proximity to these hair-cell-like cells (arrows), suggesting a divisional pair. Scale bars=5 μm. FIGS. 8C-8E are confocal images of three hair-cell-like cells at the level of the hair cell nuclear layer. Arrow in FIG. 8C indicates a neighboring cell that is BrdU-positive/myosin-VIIA-negative. Scale bars=5 μm.

FIGS. 9A and 9B, are a series of images demonstrating the levels of S-phase entry in the sensory epithelium of adult mouse utricles infected with adenoviruses encoding cell cycle regulators. FIG. 9A shows confocal images of cultured utricles from adult mice infected with adenoviruses encoding L-Myc, N-Myc, c-Myc, Oct3/4-K1f4-Sox2-c-Myc (OKSMpolycis), and c-MycT58A (1×10¹⁰ TU/mL). After washing out the virus, utricles were cultured for an additional 5 or 10 days post-virus (DPV) in the presence of BrdU. White dashed lines demarcate the borders of the sensory epithelium. FIG. 9B is a graph showing quantification of the mean number of BrdU-positive nuclei within the sensory epithelium of the infected utricles (n=2-4 utricles).

FIG. 10 is a series of images demonstrating that increased protein levels are detected in supporting cells infected with adenoviruses that encode c-Myc, N-Myc, and Oct3/4-Klf4-Sox2-c-Myc (OKSMpolycis), and c-MycT58A (each at 1×10¹⁰ TU/mL). Left panels are confocal images of the sensory epithelium in utricles infected with the indicated viruses and cultured for an additional 48 hrs. Antibody labeling for L-Myc, N-Myc, c-MYC, and Klf4 is shown. White dashed lines demarcate the borders of the sensory epithelium. Right panels are zoomed regions of the sensory epithelia shown on the left. Confocal sections at the level of supporting cell nuclei are shown. All nuclei are labeled with DRAQS.

FIGS. 11A and 11B, depict how supporting cells in the striola of the adult mouse utricle may be more amenable to MYC-induced cell cycle re-entry. FIG. 11A shows a confocal image of a utricle that was infected with a high concentration of Ad.MT58A (1×10¹⁰ TU/mL) and fixed 2 DPV. Antibody labeling of Ki67 is shown in green. A high density of Ki67-positive cells appears to be restricted to the striola (the crescent shaped region in the center of the sensory epithelium). The striola has previously been identified as a potential location for adult stem cells (Collado et al., 2011, J. Neurosci. 31:11855-11866). FIG. 11B shows a confocal image of a control utricle that was infected with the same concentration of Ad.GFP and fixed 2 DPV. GFP is shown in green. The density of GFP-labeling is similar throughout the sensory epithelium, indicating that the striola-specific effects with Ad.MT58A are not a result of viral transduction being limited to that region.

### DETAILED DESCRIPTION

The present invention provides methods for stimulating the formation of inner ear cells, including inner ear sensory hair cells and inner ear supporting cells. In one aspect, there is provided a method of treating or preventing hearing loss and/or balance disorders in a mammal, the method comprising administering to the mammal in need thereof, an expression vector encoding a gene of the present invention. Preferably, the gene of interest is expressed in a cell of an inner ear organ, or associated neural structures, of the mammal so as to treat or prevent hearing loss.

In one embodiment, the inventive provides a method of promoting the generation of sensory hair cells that allow perception of stimuli. Accordingly, the inventive method prophylactically or therapeutically treats a mammal for at least one disorder associated with loss, damage, absence of sensory hair cells, such as hearing loss and balance disorders. Hearing loss and balance disorders can be caused by damage of hair cells of the organ of Corti or vestibular organs, respecitvely, as a result of bacterial or viral infection, heredity, physical injury, acoustic trauma, and the like. While hearing loss is easily identified, balance disorders manifest in a broad variety of complications easily attributable to other ailments. Symptoms of a balance disorder include disorientation, dizziness, vertigo, nausea, blurred vision, clumsiness, and frequent falls. Balance disorders treated by the inventive method preferably involve a peripheral vestibular disorder (i.e., a disturbance in the vestibular apparatus) involving dysfunctional translation of mechanical stimuli into neural impulses due to damage or lack of sensory hair cells.

In one embodiment, the present invention provides methods for stimulating the formation of inner ear sensory hair cells from inner ear supporting cells. In one embodiment, the step of stimulating the formation of one or more inner ear sensory hair cells from one or more inner ear supporting cells includes the step of stimulating the inner ear supporting cells to enter the cell cycle, then stimulating at least some of the progeny of the inner ear supporting cells to differentiate to form inner ear sensory hair cells. In one embodiment, the step of stimulating the formation of one or more inner ear sensory hair cells from one or more inner ear supporting cells includes the step of stimulating the de-differentiation of inner ear supporting cells to a multipotent state, then stimulating at least some of the multipotent cells to differentiate to form inner ear sensory hair cells.

In one embodiment, supporting cells and hair cells can be generated by administering to the inner ear an expression vector (e.g., a viral vector) comprising a nucleic acid sequence encoding a gene of the invention including but is not limited to the Myc family members. Preferably, the gene is c-Myc. The nucleic acid sequence is expressed to produce the cell cycle re-entry protein of the invention, which results in the cell to divide. In some instances, it may be desirable to provide a protective or survival agent to promote the survival of the induced cells. In some instances, it may be desirable to provide a differentiation agent to the cells that re-enter the cell cycle to induce the cells to differentiate into the desired cell type.

In one embodiment, supporting cells can be de-differentiated by administering to the inner ear an expression vector (e.g., a viral vector) comprising a nucleic acid sequence encoding a gene of the invention including but is not limited to a transcription factor expressed in stem cells, such as c-Myc, Oct3/4, Klf4, or any combination thereof. The nucleic acid sequence is expressed to produce the de-differentiation protein of the invention, which results in the cell becoming multipotent (i.e. a cell that is not a hair cell or supporting cell but is capable of differentiating into a hair cell or supporting cell). In some instances, it may be desirable to provide a protective or survival agent to promote the survival of the induced cells. In some instances, it may be desirable to provide a differentiation agent to the cells that de-differentiate to induce the cells to differentiate into the desired cell type.

In one embodiment, the invention includes the stimulation of inner ear sensory epithelium regeneration via the delivery of an exogenous gene selected from the group consisting of c-Myc, L-Myc, N-Myc, Oct3/4, Klf4, Atoh1, Sox2, Pax2, Eya1, Six1, and any combination thereof to the cells in the sensory epithelium in situ. Exogenous delivery of the genes of the invention results in an increase in the products of those genes at the transcript or protein level.

In some embodiments, inducing the expression of for example, c-Myc, in the cells comprises inducing the expression of exogenous c-Myc in the cells, e.g., by transducing the cells with a vector encoding c-Myc, e.g., a plasmid vector or a viral vector, e.g., an adenovirus, adeno-associated virus, lentivirus, or retrovirus. Preferably, the cell cycle re-entry gene of the present invention is expressed by an adenovirus vector or an adeno-associated virus vector. However, the invention should not be limited to c-Myc. Rather, the invention includes other Myc family members, related proteins, and upstream or downstream targets in the Myc signaling pathway. A non-limiting example of such members and proteins include N-Myc, c-Myc, cyclin D1, Erk, PI3K, NMI, and ARF.

In some instances, it is desirable to enhance the stability of the protein that induces cell cycle re-entry. In one embodiment, enhanced protein stability can be achieved by modifying the nucleic acid sequence encoding the protein. This is because the invention is based partly on the discovery that the T58A mutant of c-Myc, which enhances c-Myc protein stability, is able to induce much higher levels of S-phase entry than wild-type c-Myc. However, the invention should not be limited to the T58A mutant of c-Myc. Rather, any method that stabilizes a Myc family member or upstream or downstream components of the Myc signaling pathway are included in the present invention.

In some instances, it is desirable to inhibit the functionality of genes that limit cell cycle progression after cell cycle re-entry. These genes include but are not limited to NMI and ARF, which are known to induce cell cycle arrest in response to elevated levels of c-Myc. In one embodiment, cell cycle progression can be maximized by administering to the inner ear an inhibitor that reduces the functionality of such a gene (e.g. NMI or ARF) or its products.

In some instances, it is desirable to only stimulate cell cycle re-entry within a subpopulation of cells within the inner ear sensory epithelium. In one embodiment, expression from a vector that induces cell cycle re-entry is controlled by inserting supporting-cell-specific promoter regions into the vector (e.g. promoter regions from the Sox2, Sox9, or Sox11 genes). In one embodiment, expression from such a vector occurs within supporting cells. This prevents surviving hair cells from re-entering the cell cycle, which compromises their function. In one embodiment, expression from a vector that induces cell cycle re-entry is controlled by inserting inner-ear-stem-cell specific promoter regions into the vector (e.g. promoter regions from the Lgr5 gene, or promoter regions from genes that are specifically expressed within supporting cells of the utricular striola). In one embodiment, expression from such a vector occurs within cells that are less differentiated than others within the sensory epithelium. This is because less differentiated cells within the sensory epithelium may be more likely to re-enter the cell cycle.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Allogeneic” refers to a graft derived from a different animal of the same species.

As used herein, the term “associated neural structures” when used in conjunction with the inner ear cell associated neural structures, is intended to mean the neural processes, both efferent and afferent that contact or influence the inner ear hair cell function and transmit hair cell activity centrally to the brain, or from the brain to the inner ear.

As used herein, the term “autologous” is meant to refer to any material derived from the same individual.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein the term “hearing loss” is intended to mean any reduction in a subject's ability to detect sound. Hearing loss is defined as a 10 decibel (dB) standard threshold shift or greater in hearing sensitivity for two of 6 frequencies ranging from 0.5-6.0 (0.5, 1, 2, 3, 4, and 6) kHz (cited in Dobie, R. A. (2005) Audiometric Threshold Shift Definitions: Simulations and Suggestions, Ear and Hearing 26(1) 62-77). Hearing loss can also be only high frequency, and in this case would be defined as 5 dB hearing loss at two adjacent high frequencies (2-6 kHz), or 10 dB at any frequency above 2 kHz. One example of hearing loss is age-related (or aging-related) hearing loss, which is the gradual onset of hearing loss with increasing age.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

As used herein, the phrase “improving auditory function” or “improvement in auditory function”, or grammatical equivalents thereof, means improving, by at least 10%, the sensitivity to sound of an inner ear by treating the inner ear in accordance with the methods of the present invention, or effecting any measurable improvement in the sensitivity to sound of an inner ear that is completely unresponsive to sound prior to treatment in accordance with the present invention. The sensitivity to sound of the treated inner ear is measured by any art-recognized means (such as the auditory brainstem response) and compared to the sensitivity to sound of a control inner ear that is not treated in accordance with the present invention and which is cultured under substantially the same conditions as the treated inner ear.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced into a cell of such organism in vivo.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Xenogeneic” refers to a graft derived from an animal of a different species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based partly on the discovery that inducing cell cycle re-entry in situ with exogenous delivery of c-Myc provides several advantages including but not limited to: 1) stem cells do not have to be delivered to the inner ear to restore the total number of hair cells and supporting cells to healthy levels; 2) the in situ environment may contain sufficient cues for directing the induced cells to either a supporting cell or hair cell fate, and if not, the induced cells may be exogenously manipulated to adopt the appropriate cell fate; and 3) the in situ environment may contain sufficient cues for directing the induced cells into the correct patterns and positions. However, the invention should not be limited to c-Myc. Rather, the invention includes other Myc family members, related proteins, and upstream or downstream components in the Myc signaling pathway. A non-limiting example of such members and proteins include N-Myc, c-Myc, cyclin D1, Erk, PI3K, NMI, and ARF.

In one embodiment, the invention provides a method that can be used to induce cells of the inner ear to re-enter the cell cycle. These cells that have re-entered the cycle either spontaneously differentiate into the correct ear cell types (e.g., hair cells and supporting cells), or they can be induced to do so.

In one embodiment, the invention is directed to a method comprising administering an expression vector comprising a nucleic acid sequence encoding a transcription factor including but is not limited to c-Myc, L-Myc, N-Myc, Oct3/4, Klf4, Atoh1, Sox2, Pax2, Eya1, Six1, or any combination thereof. Preferably, the expression vector is an adenoviral or adenovirus-associated vector that can express the desired gene of the invention in a cell of an inner ear organ, or associated neural structures. However, the invention is not limited to using DNA delivery schemes. Rather, any delivery schemes can be used including but not limited to RNA delivery and protein delivery schemes.

In one embodiment, the invention provides introducing c-Myc to the inner ear to promote generation of hair cells in vivo. This is because the invention is based partly on the discovery that c-Myc alone is sufficient to induce S-phase entry in supporting cells, and the supporting cells that have re-entered the cell cycle are capable of becoming new hair cells, either spontaneously or through an agent that promotes differentiation. Accordingly, the invention provides compositions and methods of increasing c-Myc expression in supporting cells for the generation of new hair cells. However, the invention should not be limited to c-Myc. Rather, any c-Myc related genes are contemplated in the invention. For example, Myc family members (e.g., N-Myc and L-Myc) and upstream and downstream components of the Myc family member pathways are included in the present invention.

In one embodiment, any agent that is able to increase c-Myc expression in supporting cells can be used to generate hair cells. An example of such an agent is a pharmacological agent that stimulates c-Myc. Exemplary pharmacological agents include but are not limited to activators of protein kinase C such as phorbol ester analogues, bombesin analogues, forskolin, sulindac, and the like.

In some instances, it is desirable to provide a protective or survival agent to promote the survival of the induced cells. In one embodiment, protection or survival can be achieved by administering to the inner ear an expression vector (e.g., a viral vector) comprising a nucleic acid sequence encoding a dominant-negative mutated form of a gene that promotes cellular damage or death (e.g. p53). The nucleic acid sequence is expressed to produce the dominant-negative mutated form of the protection or survival protein, which results in the cell escaping damage or death. In one embodiment, protection of survival can be achieved by administering to the inner ear an inhibitor consisting of but not limited to a small interfering RNA (siRNA), a microRNA, an antisense nucleic acid, or a ribozyme that in some way reduces the functionality of a gene or its products (e.g. p53) that promotes cellular damage or death. In one embodiment, protection or survival can be induced using pharmacological compounds, such as but not limited to drugs that target caspases of the apoptosis pathway (e.g. carbobenzoxy-valyl-alanyl-aspartyl-[O-methyl]-fluoromethylketone, a.k.a Z-VAD-FMK). Delivery of the protection or survival agent can occur prior, in conjunction with, or subsequent to delivery of the cell cycle re-entry agent.

In some instances, it is desirable to provide a differentiation agent to cells that have re-entered the cell cycle in order to promote differentiation of targeted cells into appropriate inner ear cell types (e.g. supporting cells and hair cells). In one embodiment, differentiation can be achieved by administering to the inner ear an expression vector (e.g., a viral vector) comprising a nucleic acid sequence encoding a gene including but is not limited to Sox2 (supporting cells), Atoh1 (hair cells), Eya1 (hair cells), Six1 (hair cells), or some combination thereof. The nucleic acid sequence is expressed to produce the differentiation protein, which results in the cell to adopt the appropriate phenotype. In one embodiment, differentiation can be induced by delivery of xenogeneic material isolated from the inner ear of non-mammals. Such xenogeneic material can consist of any biological entity, e.g. extracellular matrix components from the utricular stroma of chickens. In one embodiment, differentiation can be induced using pharmacological compounds, such as but not limited to drugs that target the Notch signaling pathway (e.g. N-[N-(3,5-Difluorophenacetyl-L-alanyl)]-S-phenylglycine t-Butyl Ester, a.k.a. DAPT or gamma secretase inhibitor IX). Delivery of the differentiation agent can occur prior, in conjunction with, or subsequent to delivery of the cell cycle re-entry agent.

In one embodiment, the invention provides compositions and methods for modulating gene expression in a target ear cell. This is because in one embodiment of the invention, expression of c-Myc induces cells to re-enter the cell cycle. However, it may be desirable to subsequently decrease the expression of the gene of the invention in the modified cell in order to have the cell differentiate into a desired cell type. Gene expression can be engineered to be turned-off using appropriate expression vectors. Alternatively, the expression of a gene can be decreased or its function blocked using pharmacological compounds, such as the c-Myc inhibitors, (Z,E)-5-(4-Ethylbenzylidine)-2-thioxothiazolidin-4-one and Int-H1-S6A,F8A.

### Vectors

One aspect of the present invention is a vector that comprises at least one gene that induces cells in the ear to re-enter the cycle and divide. As used herein, the genes of the present invention that induces ear cells to re-enter the cell cycle encode a polypeptide referred to as cell cycle re-entry proteins.

In brief summary, the expression of natural or synthetic nucleic acids associated with the genes of the present invention is typically achieved by operably linking a nucleic acid encoding the cell cycle re-entry protein or portions thereof to one or more promoters, and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

The expression constructs of the present invention may also be used for gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno- associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. One example of an element known to increase expression levels is the woodchuck hepatitis post-transcriptional regulatory element (WPRE) (see, for example, Klein et al., 2006, Gene 372:153-161).

In order to assess the expression of a cell cycle re-entry polypeptide or portions thereof, the expression vector to be introduced into a cell can also contain either a selectable marker gene or a reporter gene or both to facilitate identification and selection of expressing cells from the population of cells sought to be transfected or transduced through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter—˜driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Gene Therapy

The invention features a method of treating human patients with hearing loss using a cell cycle re-entry gene of the present invention. The cell cycle re-entry gene of the present invention can be administered through gene therapy using an adenoviral expression vector encoding the cell cycle re-entry protein, in which the gene is positioned in the vector for expression in the cells of the inner ear organ. However, the invention is not limited to using DNA delivery schemes. Rather, any delivery schemes can be used including but not limited to RNA delivery and protein delivery schemes.

In some instances, the invention includes target the expression of a desired gene to supporting cells. In one embodiment, it is desirable to target the expression of the gene to supporting cells without targeting the expression of the gene in hair cells. In yet another embodiment, it is desirable to target the expression of the gene to a potential stem cell population in the sensory epithelium, such as a stem cell population that is marked by Lgr5.

Generally speaking, the inner ear organ includes both the hearing and the vestibular organs (including the semicircular canals and the otolith organs (utricle and saccule). These organs have cells, which include 1) hearing related sensory hair cells and supporting cells, including outer hair cells; 2) sensory hair cells and supporting cells and matrix and mechanical structures for sensing vestibular function (both rotation, linear motion and gravity); and 3) associated neural structures and spiral ganglion cells.

In addition to age-related hearing loss, the present invention also contemplates that other types of hearing loss may be treatable using the expression vector described herein. Examples of other types of hearing loss include, for example: 1) ototoxicity caused by chemical or pharmaceutical agents, for example, antineoplastic agents such as cisplatinum or related compounds, aminoglycosides, antineoplastic agents, and other chemical ototoxic agents; 2) noise induced hearing loss, either from acoustic trauma or blast injury; 3) therapeutic radiation; 4) viral infections of the inner ear, such as Herpes Simplex or other viruses or infectious agents (such as Lyme Disease) that can cause inner ear hearing loss; 5) autoimmune inner ear diseases; 6) genetic hearing losses that may have an apoptotic component; 7) inner ear barotrauma such as diving or acute pressure changes; 8) physical trauma such as that caused by head injury, or surgical trauma from surgical intervention in the inner ear; and 9) inflammation or other response to administration of other inner ear regenerative compounds or gene therapy techniques.

In general, there are two approaches to gene therapy in humans. For in vivo gene therapy, a vector encoding the gene of interest can be administered directly to the patient. Alternatively, in ex vivo gene therapy, cells are removed from the patient and treated with a vector to express the gene of interest. In the ex vivo method of gene therapy, the treated cells are then re-administered to the patient.

Numerous different methods for gene therapy are well known in the art. These methods include, but are not limited to, the use of DNA plasmid vectors as well as DNA and RNA viral vectors.

One of ordinary skill in the art will appreciate that any of a number of expression vectors known in the art are suitable for introducing the nucleic acid sequence to the inner ear. Examples of suitable expression vectors include, for instance, plasmids, plasmid-liposome complexes, and viral vectors, e.g., parvoviral-based vectors (i.e., adeno-associated virus (AAV)-based vectors), retroviral vectors, herpes simplex virus (HSV)-based vectors, AAV-adenoviral chimeric vectors, and adenovirus-based vectors. Any of these expression vectors can be prepared using standard recombinant DNA techniques described in, e.g., Sambrook et al., Molecular Cloning, A Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

AAV vectors are viral vectors of particular interest for use in gene therapy protocols. AAV is a DNA virus, which is not known to cause human disease. AAV requires co-infection with a helper virus (i.e., an adenovirus or a herpes virus), or expression of helper genes, for efficient replication. AAV vectors used for administration of a therapeutic nucleic acid have approximately 96% of the parental genome deleted, such that only the terminal repeats (ITRs), which contain recognition signals for DNA replication and packaging, remain. This eliminates immunologic or toxic side effects due to expression of viral genes. Host cells comprising an integrated AAV genome show no change in cell growth or morphology (see, for example, U.S. Pat. No. 4,797,368). Although efficient, the need for helper virus or helper genes can be an obstacle for widespread use of this vector.

Retrovirus is an RNA virus capable of infecting a wide variety of host cells. Upon infection, the retroviral genome integrates into the genome of its host cell and is replicated along with host cell DNA, thereby constantly producing viral RNA and any nucleic acid sequence incorporated into the retroviral genome. When employing pathogenic retroviruses, e.g., human immunodeficiency virus (HIV) or human T-cell lymphotrophic viruses (HTLV), care must be taken in altering the viral genome to eliminate toxicity. A retroviral vector can additionally be manipulated to render the virus replication-incompetent. As such, retroviral vectors are thought to be particularly useful for stable gene transfer in vivo. Lentiviral vectors, such as HIV-based vectors, are exemplary of retroviral vectors used for gene delivery. Unlike other retroviruses, HIV-based vectors are known to incorporate their passenger genes into non-dividing cells and, therefore, are particularly useful in the sensory epithelium of the inner ear where sensory cells do not regenerate.

HSV-based viral vectors are suitable for use as an expression vector to introduce nucleic acids into the inner ear for transduction of target cells. The mature HSV virion consists of an enveloped icosahedral capsid with a viral genome consisting of a linear double-stranded DNA molecule that is 152 kb. Most replication-deficient HSV vectors contain a deletion to remove one or more intermediate-early genes to prevent replication. Advantages of the herpes vector are its ability to enter a latent stage that can result in long-term DNA expression, and its large viral DNA genome that can accommodate exogenous DNA up to 25 kb. Of course, this ability is also a disadvantage in terms of short-term treatment regimens. For a description of HSV-based vectors appropriate for use in the inventive methods, see, for example, U.S. Pat. Nos. 5,837,532, 5,846,782, 5,849,572, and 5,804,413, and International Patent Applications WO 91/02788, WO 96/04394, WO 98/15637, and WO 99/06583.

Adenovirus (Ad) is a 36 kb double-stranded DNA virus that efficiently transfers DNA in vivo to a variety of different target cell types. For use in the inventive method, the virus is preferably made replication-deficient by deleting select genes required for viral replication. The expendable non-replication-essential E3 region is also frequently deleted to allow additional room for a larger DNA insert. The vector can be produced in high titers and can efficiently transfer DNA to replicating and non-replicating cells. Genetic information transferred to a cell by way of an adenoviral vector remains epi-chromosomal, thus eliminating the risks of random insertional mutagenesis and permanent alteration of the genotype of the target cell. However, if desired, the integrative properties of AAV can be conferred to adenovirus by constructing an AAV-Ad chimeric vector. For example, the AAV ITRs and nucleic acid encoding the Rep protein incorporated into an adenoviral vector enables the adenoviral vector to integrate into a mammalian cell genome. Therefore, AAV-Ad chimeric vectors are an interesting option for use in the context of the invention.

Preferably, the expression vector of the inventive method is a viral vector; more preferably, the expression vector is an adenoviral vector. Adenovirus from any origin, any subtype, mixture of subtypes, or any chimeric adenovirus can be used as the source of the viral genome for the adenoviral vector of the invention. A human adenovirus preferably is used as the source of the viral genome for the replication-deficient adenoviral vector. The adenovirus can be of any subgroup or serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Preferably, the adenoviral vector is of subgroup C, especially serotype 2 or even more desirably serotype 5.

However, non-group C adenoviruses, and even non-human adenoviruses, can be used to prepare replication-deficient adenoviral gene transfer vectors for delivery of DNA to target cells in the inner ear. Preferred adenoviruses used in the construction of non-group C adenoviral gene transfer vectors include Ad12 (group A), Ad7 and Ad35 (group B), Ad30 and Ad36 (group D), Ad4 (group E), and Ad41 (group F). Non-group C adenoviral vectors, methods of producing non-group C adenoviral vectors, and methods of using non-group C adenoviral vectors are disclosed in, for example, U.S. Pat. Nos. 5,801,030, 5,837,511, and 5,849,561, and International Patent Applications WO 97/12986 and WO 98/53087. Preferred non-human adenoviruses include, but are not limited to, simian (e.g., SAV 25), bovine, canine, porcine adenoviruses.

The adenoviral vector is preferably replication-deficient. By “replication-deficient” is meant that the adenoviral vector comprises an adenoviral genome that lacks at least one replication-essential gene function (i.e., such that the adenoviral vector does not replicate in typical host cells, especially those in the human patient that could be infected by the adenoviral vector in the course of treatment in accordance with the invention). A deficiency in a gene, gene function, or gene or genomic region, as used herein, is defined as a deletion of sufficient genetic material of the viral genome to impair or obliterate the function of the gene whose nucleic acid sequence was deleted in whole or in part. While deletion of genetic material is preferred, mutation of genetic material by addition or substitute also is appropriate for disrupting gene function. Replication-essential gene functions are those gene functions that are required for replication (e.g., propagation) and are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA1 and/or VA-RNA-2). More preferably, the replication-deficient adenoviral vector comprises an adenoviral genome deficient in at least one replication-essential gene function of one or more regions of the adenoviral genome. Preferably, the adenoviral vector is deficient in at least one gene function of the E1 region or the E4 region of the adenoviral genome required for viral replication (denoted an E1-deficient adenoviral vector or an E4-deficient adenoviral vector). In addition to a deficiency in the E1 region, the recombinant adenovirus also can have a mutation in the major late promoter (MLP), as discussed in International Patent Application WO 00/00628. Most preferably, the adenoviral vector is deficient in at least one replication-essential gene function (desirably all replication-essential gene functions) of the E1 region and at least part of the nonessential E3 region (e.g., an Xba I deletion of the E3 region) (denoted an E1/E3-deficient adenoviral vector). With respect to the E1 region, the adenoviral vector can be deficient in part or all of the E1A region and part or all of the E1B region, e.g., in at least one replication-essential gene function of each of the E1A and E1B regions. When the adenoviral vector is deficient in at least one replication-essential gene function in one region of the adenoviral genome (e.g., an E1- or E1/E3-deficient adenoviral vector), the adenoviral vector is referred to as “singly replication-deficient.”

The adenoviral vector of the invention can be “multiply replication-deficient,” meaning that the adenoviral vector is deficient in one or more replication-essential gene functions in each of two or more regions of the adenoviral genome. For example, the aforementioned E1-deficient or E1/E3-deficient adenoviral vector can be further deficient in at least one replication-essential gene function of the E4 region (denoted an E1/E4- or E1/E3/E4-deficient adenoviral vector), and/or the E2 region (denoted an E1/E2- or E1/E2/E3-deficient adenoviral vector), preferably the E2A region (denoted an E1/E2A- or E1/E2A/E3-deficient adenoviral vector). Ideally, the adenoviral vector lacks replication-essential gene functions of only those replication-essential gene functions encoded by the early regions of the adenoviral genome, although this is not required in all contexts of the invention. A preferred multiply-deficient adenoviral vector comprises an adenoviral genome having deletions of nucleotides 457-3332 of the E1 region, nucleotides 28593-30470 of the E3 region, nucleotides 32826-35561 of the E4 region, and, optionally, nucleotides 10594-10595 of the region encoding VA-RNA1. However, other deletions may be appropriate. Nucleotides 356-3329 or 356-3510 can be removed to create a deficiency in replication-essential E1 gene functions. Nucleotides 28594-30469 can be deleted from the E3 region of the adenoviral genome. While the specific nucleotide designations recited above correspond to the adenoviral serotype 5 genome, the corresponding nucleotides for non-serotype 5 adenoviral genomes can easily be determined by those of ordinary skill in the art.

The adenoviral vector, when multiply replication-deficient, especially in replication-essential gene functions of the E1 and E4 regions, preferably includes a spacer element to provide viral growth in a complementing cell line similar to that achieved by singly replication-deficient adenoviral vectors, particularly an E1-deficient adenoviral vector. The spacer element can contain any sequence or sequences which are of a desired length, such as sequences at least about 15 base pairs (e.g., between about 15 base pairs and about 12,000 base pairs), preferably about 100 base pairs to about 10,000 base pairs, more preferably about 500 base pairs to about 8,000 base pairs, even more preferably about 1,500 base pairs to about 6,000 base pairs, and most preferably about 2,000 to about 3,000 base pairs in length. The spacer element sequence can be coding or non-coding and native or non-native with respect to the adenoviral genome, but does not restore the replication-essential function to the deficient region. The use of a spacer in an adenoviral vector is described in U.S. Pat. No. 5,851,806. In one embodiment of the inventive method, the replication-deficient or conditionally-replicating adenoviral vector is an E1/E4-deficient adenoviral vector wherein the L5 fiber region is retained, and a spacer is located between the L5 fiber region and the right-side ITR. More preferably, in such an adenoviral vector, the E4 polyadenylation sequence alone or, most preferably, in combination with another sequence, exists between the L5 fiber region and the right-side ITR, so as to sufficiently separate the retained L5 fiber region from the right-side ITR, such that viral production of such a vector approaches that of a singly replication-deficient adenoviral vector, particularly an E1-deficient adenoviral vector.

The adenoviral vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case it is preferred that at least either the viral inverted terminal repeats (ITRs) and one or more promoters or the viral ITRs and a packaging signal are left intact (i.e., an adenoviral amplicon). The 5′ or 3′ regions of the adenoviral genome comprising ITRs and packaging sequence need not originate from the same adenoviral serotype as the remainder of the viral genome. For example, the 5′ region of an adenoviral serotype 5 genome (i.e., the region of the genome 5′ to the adenoviral E1 region) can be replaced with the corresponding region of an adenoviral serotype 2 genome (e.g., the Ad5 genome region 5′ to the E1 region of the adenoviral genome is replaced with nucleotides 1-456 of the Ad2 genome). Suitable replication-deficient adenoviral vectors, including multiply replication-deficient adenoviral vectors, are disclosed in U.S. Pat. Nos. 5,837,511, 5,851,806, and 5,994,106, U.S. Published Patent Applications 2001/0043922 A1 2002/0004040 A1, 2002/0031831 A1, and 2002/0110545 A1, and International Patent Applications WO 95/34671, WO 97/12986, and WO 97/21826. Ideally, the replication-deficient adenoviral vector is present in a pharmaceutical composition virtually free of replication-competent adenovirus (RCA) contamination (e.g., the pharmaceutical composition comprises less than about 1% of RCA contamination). Most desirably, the pharmaceutical composition is RCA-free. Adenoviral vector compositions and stocks that are RCA-free are described in U.S. Pat. Nos. 5,944,106 and 6,482,616, U.S. Published Patent Application 2002/0110545 A1, and International Patent Application WO 95/34671.

Therefore, in a preferred embodiment, the expression vector of the inventive method is a multiply replication-deficient adenoviral vector lacking all or part of the E1 region, all or part of the E3 region, all or part of the E4 region, and, optionally, all or part of the E2 region. It is believed that multiply deficient vectors are particularly suited for delivery of exogenous nucleic acid sequences to the ear. Adenoviral vectors deficient in at least one replication-essential gene function of the E1 region are most commonly used for gene transfer in vivo. However, currently used singly replication-deficient adenoviral vectors can be detrimental to the sensitive cells of the epithelium of the inner ear, causing damage to the very cells to be treated. Adenoviral vectors that are deficient in at least one replication-essential gene function of the E4 region, particularly adenoviral vectors deficient in replication-essential gene functions of the E4 region and the E1 region, are less toxic to cells than E1-deficient adenoviral vectors (see, for example, Wang et al., Nature Medicine, 2(6), 714-716 (1996) and U.S. Pat. No. 6,228,646). Accordingly, damage to existing hair cells and supporting cells can be minimized by employing an E1,E4-deficient adenoviral vector to deliver the nucleic acid sequence encoding the cell cycle re-entry protein of the invention to inner ear cells.

Replication-deficient adenoviral vectors are typically produced in complementing cell lines that provide gene functions not present in the replication-deficient adenoviral vectors, but required for viral propagation, at appropriate levels in order to generate high titers of viral vector stock. A preferred cell line complements for at least one and preferably all replication-essential gene functions not present in a replication-deficient adenovirus. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons). Most preferably, the complementing cell line complements for a deficiency in at least one replication-essential gene function (e.g., two or more replication-essential gene functions) of the E1 region of the adenoviral genome, particularly a deficiency in a replication-essential gene function of each of the E1A and E1B regions. In addition, the complementing cell line can complement for a deficiency in at least one replication-essential gene function of the E2 (particularly as concerns the adenoviral DNA polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORF of the E4 region of the adenoviral genome. The cell line preferably is further characterized in that it contains the complementing genes in a non-overlapping fashion with the adenoviral vector, which minimizes, and practically eliminates, the possibility of the vector genome recombining with the cellular DNA. Accordingly, the presence of replication competent adenoviruses (RCA) is minimized if not avoided in the vector stock, which, therefore, is suitable for certain therapeutic purposes, especially gene therapy purposes. The lack of RCA in the vector stock avoids the replication of the adenoviral vector in non-complementing cells. Construction of such a complementing cell lines involve standard molecular biology and cell culture techniques, such as those described by Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Complementing cell lines for producing the adenoviral vector include, but are not limited to, 293 cells (described in, e.g., Graham et al., J. Gen. Virol., 36, 59-72 (1977)), PER.C6 cells (described in, e.g., International Patent Application WO 97/00326, and U.S. Pat. Nos. 5,994,128 and 6,033,908), and 293-ORF6 cells (described in, e.g., International Patent Application WO 95/34671 and Brough et al., J. Virol., 71, 9206-9213 (1997)). In some instances, the complementing cell will not complement for all required adenoviral gene functions. Helper viruses can be employed to provide the gene functions in trans that are not encoded by the cellular or adenoviral genomes to enable replication of the adenoviral vector. Adenoviral vectors can be constructed, propagated, and/or purified using the materials and methods set forth, for example, in U.S. Pat. Nos. 5,965,358, 5,994,128, 6,033,908, 6,168,941, 6,329,200, 6,383,795, 6,440,728, 6,447,995, and 6,475,757, U.S. Patent Application Publication No. 2002/0034735 A1, and International Patent Applications WO 98/53087, WO 98/56937, WO 99/15686, WO 99/54441, WO 00/12765, WO 01/77304, and WO 02/29388, as well as the other references identified herein. Non-group C adenoviral vectors, including adenoviral serotype 35 vectors, can be produced using the methods set forth in, for example, U.S. Pat. Nos. 5,837,511 and 5,849,561, and International Patent Applications WO 97/12986 and WO 98/53087. Moreover, numerous adenoviral vectors are available commercially.

The adenoviral vector's coat protein can be modified so as to decrease the adenoviral vector's ability or inability to be recognized by a neutralizing antibody directed against the wild-type coat protein. Such modifications are useful for multiple rounds of administration. Similarly, the coat protein of the adenoviral vector can be manipulated to alter the binding specificity or recognition of the adenoviral vector for a viral receptor on a potential host cell. Such manipulations can include deletion or substitution of regions of the fiber, penton, hexon, pIIIa, pVI, and/or pIX, insertions of various native or non-native ligands into portions of the coat protein, and the like. Manipulation of the coat protein can broaden the range of cells infected by the adenoviral vector or enable targeting of the adenoviral vector to a specific cell type. The ability of an adenoviral vector to recognize a potential host cell can be modulated without genetic manipulation of the coat protein, i.e., through use of a bi-specific molecule. For instance, complexing an adenovirus with a bispecific molecule comprising a penton base- or fiber-binding domain and a domain that selectively binds a particular cell surface binding site enables the targeting of the adenoviral vector to a particular cell type.

Preferably, the adenoviral capsid is modified to display a non-native amino acid sequence. The non-native amino acid sequence can be inserted into or in place of an internal coat protein sequence (e.g., within an exposed loop of an adenoviral fiber protein) or fused to the terminus of an adenoviral coat protein (e.g., fused to the C-terminus of an adenoviral fiber protein, optionally using a linker or spacer sequence). The non-native amino acid sequence can be conjugated to any of the adenoviral coat proteins to form a chimeric coat protein. Therefore, for example, the non-native amino acid sequence of the invention can be conjugated to, inserted into, or attached to a fiber protein, a penton base protein, a hexon protein, proteins IX, VI, or IIIa, etc. The sequences of such proteins, and methods for employing them in recombinant proteins, are well known in the art (see, e.g., U.S. Pat. Nos. 5,543,328; 5,559,099; 5,712,136; 5,731,190; 5,756,086; 5,770,442; 5,846,782; 5,962,311; 5,965,541; 5,846,782; 6,057,155; 6,127,525; 6,153,435; 6,329,190; 6,455,314; 6,465,253; and 6,576,456; U.S. Patent Application Publication 2001/0047081 and 2003/0099619; and International Patent

Applications WO 96/07734, WO 96/26281, WO 97/20051, WO 98/07877, WO 98/07865, WO 98/40509, WO 98/54346, WO 00/15823, WO 01/58940, and WO 01/92549). The coat protein portion of the chimeric coat protein can be a full-length adenoviral coat protein to which the ligand domain is appended, or it can be truncated, e.g., internally or at the C- and/or N-terminus. The coat protein portion need not, itself, be native to the adenoviral vector.

### Methods of Treatment

The methods described herein can be used to treat a subject who is experiencing loss or impairment of hearing, loss or impairment of balance, or injury to or loss of vestibular hair cells, neurons, supporting cells, or dark cells, in order to minimize, reduce, or completely prevent or restore, the loss of hearing, the loss of balance function or of hair cells, neurons or dark cells of the vestibular portion of the inner ear. Treatment is intended to also include the possibility of inducing, causing or facilitating regeneration of the cellular elements of the inner ear including hair cells, supporting cells, dark cells, neurons and subcellular organelles of these cells including, synapses, stereocilia bundles, kinocilia, mitochondria and other cell organelles, or mechanical and functional supporting structures such as otoconia, cupula and crista of the inner ear. Treatment is also intended to prevent recurrent degeneration after regeneration of cellular elements of the inner ear, including hair cells, supporting cells, dark cells, neurons and subcellular organelles of these cells including synapses, stereocilia bundles, kinocilia, mitochondira and other cell organelles, or mechanical and functional supporting structures such as otoconia, cupula and crista of the inner ear. Treatment is also intended to mean the partial or complete restoration of hearing or balance function regardless of the cellular mechanisms involved.

The inventive method promotes the generation of sensory hair cells that allow perception of stimuli. In one embodiment, the inventive method prophylactically or therapeutically treats a mammal for at least one disorder associated with loss, damage, absence of sensory hair cells, such as hearing loss and balance disorders. Hearing loss can be caused by damage of hair cells of the organ of Corti due to bacterial or viral infection, heredity, physical injury, acoustic trauma, and the like. While hearing loss is easily identified, balance disorders manifest in a broad variety of complications easily attributable to other ailments. Symptoms of a balance disorder include disorientation, dizziness, vertigo, nausea, blurred vision, clumsiness, and frequent falls. Balance disorders treated by the inventive method preferably involve a peripheral vestibular disorder (i.e., a disturbance in the vestibular apparatus) involving dysfunctional translation of mechanical stimuli into neural impulses due to damage or lack of sensory hair cells.

A mammal having a disorder of the inner ear, or at risk for developing such a disorder, can be treated with inner ear cells (hair cells or supporting cells) generated from the methods of the invention.

Any human experiencing or at risk for developing a hearing loss is a candidate for the treatment methods described herein. For example, the human can receive gene therapy according to the present invention to induce the generation of inner ear hair cells or supporting cells. A human having or at risk for developing a hearing loss can hear less well than the average human being, or less well than a human before experiencing the hearing loss. For example, hearing can be diminished by at least 5, 10, 30, 50% or more. The human can have sensorineural hearing loss, which results from damage or malfunction of the sensory part (the cochlea) or the neural part (the auditory nerve) of the ear, or conductive hearing loss, which is caused by blockage or damage in the outer and/or middle ear, or the human can have mixed hearing loss, which is caused by a problem in both the conductive pathway (in the outer or middle ear) and in the nerve pathway (the inner ear). An example of a mixed hearing loss is a conductive loss due to a middle-ear infection combined with a sensorineural loss due to damage associated with aging.

The subject can be deaf or have a hearing loss for any reason or as a result of any type of event. For example, a human can be deaf because of a genetic or congenital defect; for example, a human can have been deaf since birth, or can be deaf or hard-of-hearing as a result of a gradual loss of hearing due to a genetic or congenital defect. In another example, a human can be deaf or hard-of-hearing as a result of a traumatic event, such as a physical trauma to a structure of the ear, or a sudden loud noise, or a prolonged exposure to loud noises. For example, prolonged exposures to concert venues, airport runways, and construction areas can cause inner ear damage and subsequent hearing loss. A human can experience chemical-induced ototoxicity, wherein ototoxins include therapeutic drugs including antineoplastic agents, salicylates, quinines, and aminoglycoside antibiotics, contaminants in foods or medicines, and environmental or industrial pollutants. A human can have a hearing disorder that results from aging, or the human can have tinnitus (characterized by ringing in the ears).

Following administration of one or more genes of the present invention into the inner ear, the mammal can be tested for an improvement in hearing or in other symptoms related to inner ear disorders. Methods for measuring hearing are well-known and include pure tone audiometry, air conduction, and bone conduction tests. These exams measure the limits of loudness (intensity) and pitch (frequency) that a human can hear. Hearing tests in humans include behavioral observation audiometry (for infants to seven months), visual reinforcement orientation audiometry (for children 7 months to 3 years) and play audiometry for children older than 3 years. Oto-acoustic emission testing can be used to test the functioning of the cochlear hair cells, and electro-cochleography provides information about the functioning of the cochlea and the first part of the nerve pathway to the brain.

### Routes of Administration

One skilled in the art will appreciate that suitable methods of administering an expression vector, such as an adenoviral vector, to the inner ear are available. Although more than one route can be used to administer a particular expression vector, a particular route can provide a more immediate and more effective reaction than another route. Accordingly, the described routes of administration are merely exemplary and are in no way limiting.

No matter the route of administration, the expression vector of the inventive method must reach the sensory epithelium of the inner ear. The most direct routes of administration, therefore, entail surgical procedures which allow access to the interior of the structures of the inner ear. Inoculation via cochleostomy allows administration of the expression vector directly to the regions of the inner ear associated with hearing. Cochleostomy involves drilling a hole through the cochlear wall, e.g., in the otic capsule below the stapedial artery as described in Kawamoto et al., Molecular Therapy, 4(6), 575-585 (2001), and release of a pharmaceutical composition comprising the expression vector. Administration to the endolymphatic compartment is particularly useful for administering the adenoviral vector to the areas of the inner ear responsible for hearing. Alternatively, the expression vector can be administered to the semicircular canals via canalostomy. Canalostomy provides for transgene expression in the vestibular system and the cochlea, whereas cochleostomy does not provide as efficient transduction in the vestibular space. The risk of damage to cochlear function is reduced using canalostomy in as much as direct injection into the cochlear space can result in mechanical damage to hair cells (Kawamoto et al., supra). Administration procedures also can be performed under fluid (e.g., artificial perilymph), which can comprise factors to alleviate side effects of treatment or the administration procedure, such as apoptosis inhibitors or anti-inflammatories.

Another direct route of administration to the inner ear is through the round window, either by injection or topical application to the round window. Administration via the round window is especially preferred for delivering an adenoviral vector to the perilymphatic space. Transgene expression in cochlear and vestibular neurons and cochlear sensory epithelia has been observed following administration of expression vectors via the round window (Staecker et al., Acta Otolaryngol, 121, 157-163 (2001)). Surprisingly, it appears possible that uptake of expression vectors, in particular non-targeted adenoviral vectors, into cells of the inner ear is not receptor-mediated. In other words, it does not appear that adenoviral infection of cells of the inner ear is mediated by integrins. To increase transduction of cells in the organ of Corti following administration to the perilymphatic compartment, the adenoviral vector can display one or more ligands that enhance uptake of the adenoviral vector into target cells (e.g., supporting cells, cells of the stria vascularis, etc.). In this regard, the adenoviral vector can comprise one or more adenoviral coat proteins which are modified to reduce native binding (e.g., integrin-binding) and comprise a non-native amino acid sequence which enhances uptake of the adenoviral vector by target cells of the inner ear.

The expression vector (e.g., adenoviral vector) can be present in a pharmaceutical composition for administration to the inner ear. In certain cases, it may be appropriate to administer multiple applications and/or employ multiple routes, e.g., canalostomy and cochleostomy, to ensure sufficient exposure of supporting cells to the expression vector.

The expression vector can be present in or on a device that allows controlled or sustained release of the expression vector, such as an sponge, meshwork, mechanical reservoir or pump, or mechanical implant. For example, a biocompatible sponge or gelform soaked in a pharmaceutical composition comprising the expression vector is placed adjacent to the round window, through which the expression vector permeates to reach the cochlea (as described in Jero et al., supra). Mini-osmotic pumps provide sustained release of an expression vector over extended periods of time (e.g., five to seven days), allowing small volumes of composition comprising the expression vector to be administered, which can prevent mechanical damage to endogenous sensory cells. The expression vector also can be administered in the form of sustained-release formulations (see, e.g., U.S. Pat. No. 5,378,475) comprising, for example, gelatin, chondroitin sulfate, a polyphosphoester, such as bis-2-hydroxyethyl-terephthalate (BHET), or a polylactic-glycolic acid.

While not particularly preferred, the expression vector can be administered parenterally, intramuscularly, intravenously, or intraperitoneally. Preferably, any expression vector parenterally administered to a patient for generating sensory hair cells in the ear is specifically targeted to sensory epithelial cells, such as supporting cells. Desirably, the expression vector is targeted to scarred sensory epithelium to promote generation of exogenous hair cells to replace damaged endogenous hair cells. As discussed herein, an expression vector can be modified to alter the binding specificity or recognition of an expression vector for a receptor on a potential host cell. With respect to adenovirus, such manipulations can include deletion of regions of the fiber, penton, or hexon, insertions of various native or non-native ligands into portions of the coat protein, and the like. One of ordinary skill in the art will appreciate that parenteral administration can require large doses or multiple administrations to effectively deliver the expression vector to the appropriate host cells. Pharmaceutically acceptable carriers for compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4.sup.th ed., pages 622-630 (1986)). Although less preferred, the expression vector can also be administered in vivo by particle bombardment, i.e., a gene gun.

One of ordinary skill in the art also will appreciate that dosage and routes of administration can be selected to minimize loss of expression vector due to a host's immune system. For example, for contacting target cells in vivo, it can be advantageous to administer to a host a null expression vector (i.e., an expression vector not comprising the nucleic acid sequence encoding an atonal-associated factor prior to performing the inventive method. Prior administration of null expression vectors can serve to create an immunity in the host to the expression vector hinder the body's innate clearance mechanisms, thereby decreasing the amount of vector cleared by the immune system.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### MYC Gene Delivery to Adult Mouse Utricles Stimulates Proliferation of Postmitotic Supporting Cells In Vitro

Described herein are methods of reprogramming differentiated supporting cells into otic progenitors as a potential strategy for restoring regenerative potential to the ear. The inner ears of adult humans and other mammals possess a limited capacity for regenerating sensory hair cells, which can lead to permanent auditory and vestibular deficits. During development and regeneration, undifferentiated supporting cells within inner ear sensory epithelia can self-renew and give rise to new hair cells; however, these otic progenitors become depleted postnatally. Therefore, transient expression of the induced pluripotency transcription factors, Oct3/4, Klf4, Sox2, and c-Myc reprograms fibroblasts into neural progenitors under neural-promoting culture conditions. Whether ectopic expression of these factors can reverse supporting cell quiescence in whole organ cultures of adult mouse utricles was explored. Co-infection of utricles with adenoviral vectors separately encoding Oct3/4, Klf4, Sox2, and the degradation-resistant T58A mutant of c-Myc (c-MycT58A) triggered significant levels of supporting cell S-phase entry was assessed by continuous BrdU labeling. Of the four factors, c-MycT58A alone was both necessary and sufficient for the proliferative response. In contrast, supporting cells remained postmitotic after ectopic expression of the three other iPSC factors, Oct3/4, Klf4, and Sox2.

The number of BrdU-labeled cells plateaued between 5-7 days after infection, and then decreased ˜60% by 3 weeks, as many cycling cells appeared to enter apoptosis. At least a portion of the cells were able to progress into M-phase, and a small number of cells replicating their DNA were found 21 days post-virus (DPV); however, many of the cycling cells appeared to enter apoptosis between 7 and 14 DPV. Switching to differentiation-promoting culture medium at 5 days after ectopic expression of c-MycT58A temporarily attenuated the loss of BrdU-labeled cells and accompanied a very modest but significant expansion of the sensory epithelium. Switching from growth medium to serum-free differentiation medium could prevent the loss of cycling cells, but the protective effect of serum deprivation was temporary and subsided by 14 DPV. Within the protective time window, a modest but significant increase in the area of the sensory epithelium was detected at 10 DPV, and a very small number of cells that had replicated their DNA labeled with antibodies to the hair cell marker myosin VIIA. A small number of the proliferating cells in these cultures labeled for the hair cell marker, myosin VIIA, suggesting they had begun differentiating towards a hair cell fate. The results indicated that ectopic expression of c-MycT58A, which may be used in combination with methods for promoting cell survival and differentiation, may restore regenerative potential to supporting cells within the adult mammalian inner ear.

The materials and methods employed in these experiments are now described.

### Materials and Methods

Animals and Dissection of Utricles

All animal work was approved by the Animal Care and Use Committee of Wake Forest University (protocol number: A11-222). Swiss Webster mice, adults of either sex (>6 weeks old) and timed-pregnant females, were obtained from Charles River (Wilmington, Mass.). Labyrinths were dissected from temporal bones in ice-cold DMEM/F-12 (Invitrogen, Carlsbad, Calif.), the utricles were isolated, and the roof, otoconia, and nerve were mechanically removed under aseptic conditions. The dissected organs contained the entire sensory epithelium, a small portion of the surrounding non-sensory epithelium, and the underlying stromal tissue.

Organ Culture and Infection with Adenoviral Vectors

Adenoviruses containing vectors encoding Oct3/4, Klf4, Sox2, c-MycT58A, or GFP under the control of a cytomegalovirus (CMV) promoter were obtained from Stemgent (Cambridge, Mass.). To construct its adenoviruses, Stemgent uses the AdEasy adenoviral vector system, which allows for insertion of a promoter and gene of interest into the E1-, E3-deleted backbone of adenovirus serotype 5. The cDNA plasmids cloned into the viral genome as described previously (Stadfeldt et al., 2008, Science 322:945-949) and can be obtained from Addgene. The Addgene plasmid ID numbers and final concentration of adenoviruses (transduction units per mL, TU/mL) used for the co-infection experiments were: mouse Oct3/4 (ID: 19768; titer: 5×107 TU/mL), mouse Klf4 (ID: 19770; titer: 2×108 TU/mL), mouse Sox2 (ID: 19767, titer: 5×107 TU/mL), and human c-Myc T58A mutant with a hemagglutinin (HA) tag (ID: 19769, titer: 2×108 TU/mL). The stock adenovirus solution comes stored in a suspension buffer consisting of 25 mM Tris (pH 7.5), 2.5 mM MgCl₂, and 1 M NaCl. Stemgent titers its adenovirus using the immunoassay titration method.

For organ culture and adenovirus infection, dissected utricles were adhered to glass-bottom dishes (Mat-Tek, Ashland, Mass.; two utricles per dish) coated with Cell-Tak (BD Biosciences, San Jose, Calif.) as described previously (Meyers and Corwin, 2007, J. Neurosci. 27:4313-4325) and maintained at 37° C. and 5% CO₂ in 100 of growth medium consisting of DMEM/F12, 5% FBS (Invitrogen), 0.25 μg/mL Fungizone (Invitrogen), and 10 μg/mL ciprofloxacin (Bayer, Berlin, Germany). Utricles were allowed to stabilize in culture for 24 h, after which the growth medium was replaced with infection medium (DMEM/F12, 0.25 μg/mL Fungizone, and 10 μg/mL ciprofloxacin). Adenoviruses were then added at the indicated concentrations for 8 h. Following adenovirus washout, utricles were cultured in 2 mL of growth medium until fixation. To label cells in S-phase, BrdU (3 μg/mL, Sigma) was added for the periods indicated. In some instances, the growth medium was replaced with 2 mL of differentiation medium to promote hair cell differentiation. Differentiation medium was based on a described formula (Montcouquiol and Kelley, 2003, J. Neurosci. 23:9469-9478) and consisted of DMEM/F12, N2 supplement (Invitrogen), 0.25 μg/mL Fungizone, and 10 μg/mL ciprofloxacin.

Immunocytochemistry

The following antibodies were used: rabbit anti-myosin VIIA (1:200; Proteus Biosciences, Ramona, Calif.; #25-6790) and mouse anti-myosin VIIA (1:100; Developmental Studies Hybridoma Bank, Iowa City, Iowa; #MYO7A 138-1) to label hair cell soma; mouse anti-BrdU (1:50; BD Biosciences; #347580) to label cells that had incorporated BrdU during S-phase; rabbit anti-Ki67 (1:200; Thermofisher Scientific, Kalamazoo, MI; #RM-9106-S0) to label cells in the active G1, S, G2, and M phases of the cell cycle; mouse anti-phospho-histone H3 (Ser10) (PH3-Ser10; 1:200, Cell Signaling Technology, Danvers, Mass.; #9706) to label cells in M phase; rabbit anti-Oct3/4 (1:200; Santa Cruz Biotechnology, Santa Cruz, Calif.; #SC-5279); mouse anti-Klf4 (1:200;Abcam, Cambridge Mass.; #AB75486); rabbit anti-Sox2 (1:200; Millipore, Billerica, Mass.; #AB5603); mouse anti-c-Myc (1:200; Santa Cruz Biotechnology; #SC-40); mouse anti-HA (1:200; Abcam; #AB18181); and rabbit anti-activated-caspase 3 to label cells undergoing apoptosis (1:200; Abcam; #AB3623).

For immunocytochemistry, utricles were fixed in fresh 4% paraformaldehyde in phosphate-buffered saline (PBS) for 1 h at room temperature (RT). After fixation, specimens were washed in PBS then permeabilized and blocked for 1 h at RT in PBS with 0.2% Triton X-100 (PBS-T) and 10% normal goat serum (NGS; Invitrogen). Samples to be labeled with anti-BrdU were digested with DNAse I (0.5 kunitz/μL; Sigma) for 1 h at 37° C. before adding the blocking solution. Samples were then incubated in the appropriate primary antibodies in PBS-T with 2% NGS overnight, followed by 3 rinses in PBS-T and labeling with AlexaFluor-conjugated secondary antibodies (1:200, Invitrogen) in PBS-T for 3 h at RT. Where indicated, AlexaFluor-conjugated phalloidin (5 U/mL, Invitrogen) and/or DRAQS (1:1000, Cell Signaling) were included with the secondary antibodies to detect F-actin and nuclei. Utricles were rinsed in PBS 3 times and mounted in SlowFade (Invitrogen). Specimens were imaged using a Zeiss LSM 510 confocal microscope.

Quantification

To quantify the percentage of GFP-expressing cells in Ad.GFP-infected utricles, the number of GFP-positive/myosin VIIA-negative supporting cells and GFP-positive/myosin VIIA-positive hair cells in 50 μm×50 μm regions were separately counted at nine different locations spaced along the anterior-posterior axis of the medial edge, striola, and lateral edge of each utricle. The average for the nine regions was computed and then divided by previous estimates of the mean density of supporting cells and hair cells in adult mouse utricles in vivo (Kirkegaard and Nyengaard, 2005, J. Comp. Neurol. 492:132-144; Burns et al., 2012, J. Neurosci. 32:6570-6577). For total BrdU counts and cell cycle phase analysis, all BrdU-, Ki67-, and PH3-Ser10-labeled nuclei in the sensory epithelium were manually counted for each utricle using the Cell Counter plugin in ImageJ (U.S. National Institutes of Health, Bethesda, Md.). Macular area was measured by using ImageJ to trace the outline of the sensory epithelium in confocal images of utricles labeled with anti-myosin VIIA.

Statistics

OriginPro 7.5 was used to conduct sigmoidal equation fits, Student's t-tests, and one-way or two-way ANOVAs followed by Tukey's Test of Multiple Comparisons (alpha level=0.05 in all cases). All descriptive statistics are presented as mean±s.e.m. To calculate nonlinear least squares fits, OriginPro uses Levenberg-Marquardt chi-squared minimization with automatic parameter initialization. To fit equations to the data, 200 iterations of this minimization routine were performed. The following sigmoidal equation was used for the fits:

$$y = \frac{{A}_{1} - {A}_{2}}{1 + {( \frac{x}{{x}_{0}} )}^{p}} + {A}_{2} .$$

The results of the experiments are now described.

### Type 5 Adenovirus Transduces Supporting Cells and a Small Fraction of Hair Cells in Adult Mouse Utricles in Vitro

The inner ear sensory epithelium is difficult to transfect using techniques such as electroporation and lipofection (Jones et al., 2006, J. Neurosci. 26: 550-558; Woods et al., 2004, Nat Neurosci 7:1310-1318; Driver and Kelley, 2010, from Transfection of mouse cochlear explants by electroporation. Current protocols in neuroscience/editorial board, Chapter 4: Unit 4 34 31-10). Some viruses efficiently infect supporting cells and/or hair cells (Holt et al., 1999, J. Neurophysiol. 81:1881-1888; Luebke et al, 2001, Human Gene Therapy 12:773-781), and ectopic gene expression in supporting cells has been observed after infecting adult mouse utricles with type 5 adenovirus in vitro (Lin et al., 2011, J. Neurosci. 31:15329-15339; Loponen et al., 2011, PloS One 6:e27360; Brandon et al., 2012, J. Vis. Exp. (61), e3734; Laine et al., 2010, Dev. Biol. 337:134-146). To characterize the efficiency of ectopic expression versus adenovirus concentration, utricles cultured from mice>6 weeks old were infected with 10⁶-10⁹ transduction units per mL (TU/mL) of adenovirus that contained a vector encoding green fluorescent protein (Ad.GFP) under the control of a CMV promoter. The cultures were fixed at 3 days post virus (DPV), and the percentage of GFP-expressing supporting cells that did not label with antibodies for the hair cell marker myosin VIIA were quantified by sampling multiple regions within the sensory epithelium.

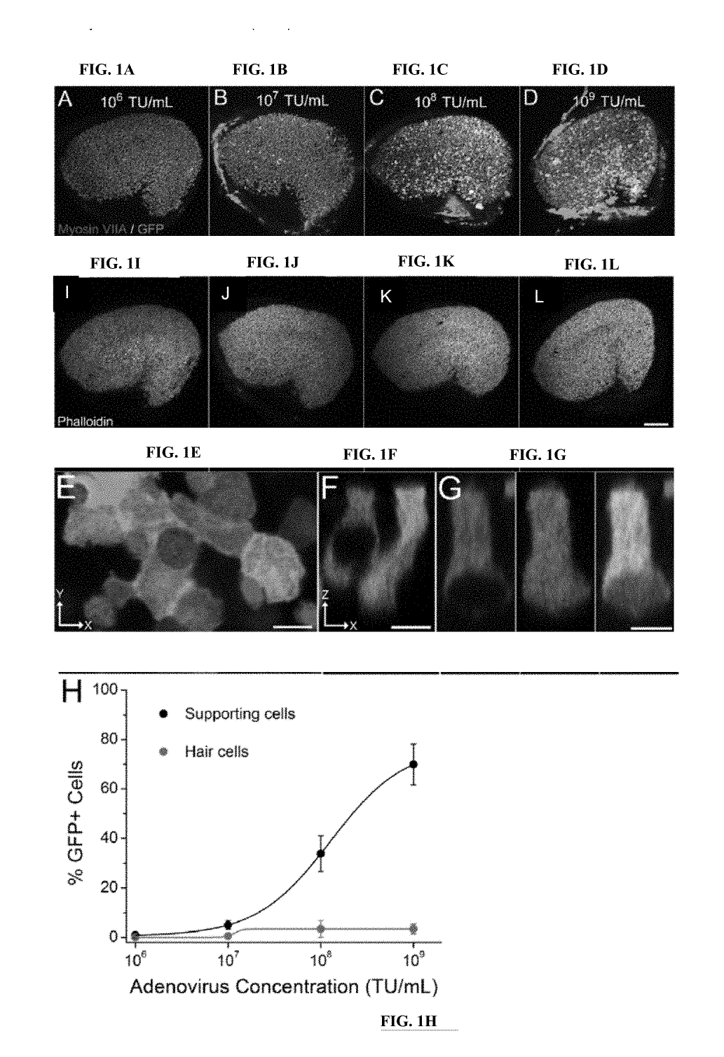

10⁶ TU/mL resulted in little to no GFP expression, but the percentage of supporting cells expressing GFP increased for concentrations ranging from 10⁷-10⁹ TU/mL (FIGS. 1A-1D, H; n=2 utricles per concentration). Adenoviral-mediated GFP expression in utricular hair cells from neonatal mice has previously been modeled with a sigmoidal function (Holt et al., 1999, J. Neurophysiol. 81:1881-1888). Nonlinear least squares fitting to this data showed that the percentage of GFP-expressing supporting cells in adult mouse utricles could similarly be modeled with a sigmoidal function (r2=1), which had a half-maximal transduction efficiency at 1.3×108 TU/mL and a maximum efficiency of 77.2% (FIG. 1H, Table 1).

TABLE 1
Coefficient Values for Sigmoidal Curve Fits

| | $y = \frac{{A}_{1} - {A}_{2}}{1 + {( \frac{x}{{x}_{0}} )}^{p}} + {A}_{2}$ | | | |
|---|---|---|---|---|
| | A₁ | A₂ | χₒ | p |
| Supporting cells | 0.5 | 77.2 | 1.3 × 10⁸ | 1.1 |
| Hair cells | 0 | 3.4 | 1.2 × 10⁷ | 10.6 |

Within the range of Ad.GFP concentrations tested, the utricular macula maintained epithelial and cytoskeletal integrity as revealed with fluorescent phalloidin labeling (FIGS. 1I-1L). The GFP-expressing supporting cells displayed typical, cylindrical shapes that were more expanded at their apical and basal ends and compacted at the level of the hair cell nuclei (FIGS. 1E-1F).

Labeling of hair cells with antibodies to myosin VIIA allowed distinguishment between hair cells and supporting cells, and it was observed that GFP expression was mostly restricted to supporting cells (FIGS. 1E-1F). However, the occasional GFP-positive/myosin VIIA-positive hair cell was with characteristic flask-like shapes was observed (FIG. 1G). The intensity of GFP-expression in these cells appeared weaker than in neighboring supporting cells, which may contribute to difficulty in identifying them since previous studies have reported that adenovirus type 5 exclusively expresses in supporting cells within adult mouse utricles (Lin et al., 2011, J. Neurosci. 1:15329-15339; Loponen et al., 2011, PloS One 6:e27360; Brandon et al., 2012, J. Vis. Exp. 61:e3734). The percentage of hair cells expressing GFP also fit with a sigmoidal function (r2=1) at virus concentrations ranging from 10⁶-10⁹ TU/mL, but the maximum hair cell transduction efficiency was only 3.4%, which was 23-times lower than for supporting cells (FIG. 1H, Table 1). This efficiency was also substantially lower than the ˜60% reported for hair cells in utricles from neonatal mice (Holt et al., 1999, J. Neurophysiol. 81:1881-1888). Also, the viral concentration yielding half-maximal hair cell transduction in adults was an order of magnitude lower than for supporting cells, but on the same order of magnitude for neonatal hair cells (FIG. 1H, Table 1; concentration at half-maximal infection=1.3×10⁷ TU/mL). Although not wishing to be bound to any particular theory, combined, the results suggest that adenovirus transduces hair cells at similar concentrations independent of age, the number of transduced hair cells decreases substantially with age, and adenovirus transduces adult hair cells at lower concentrations than adult supporting cells. It remains unclear whether the age-related decrease in GFP-expressing hair cells reflects a change in infectivity or CMV promoter activity. Although the number of GFP-expressing hair cells was relatively low, ectopic gene expression within hair cells may need to be taken into consideration when infecting adult mouse utricles with adenovirus in vitro.

Adenoviral Gene Delivery of iPSC Transcription Factors to the Adult Mouse Utricle Initiates S-Phase Entry in Postmitotic Supporting Cells

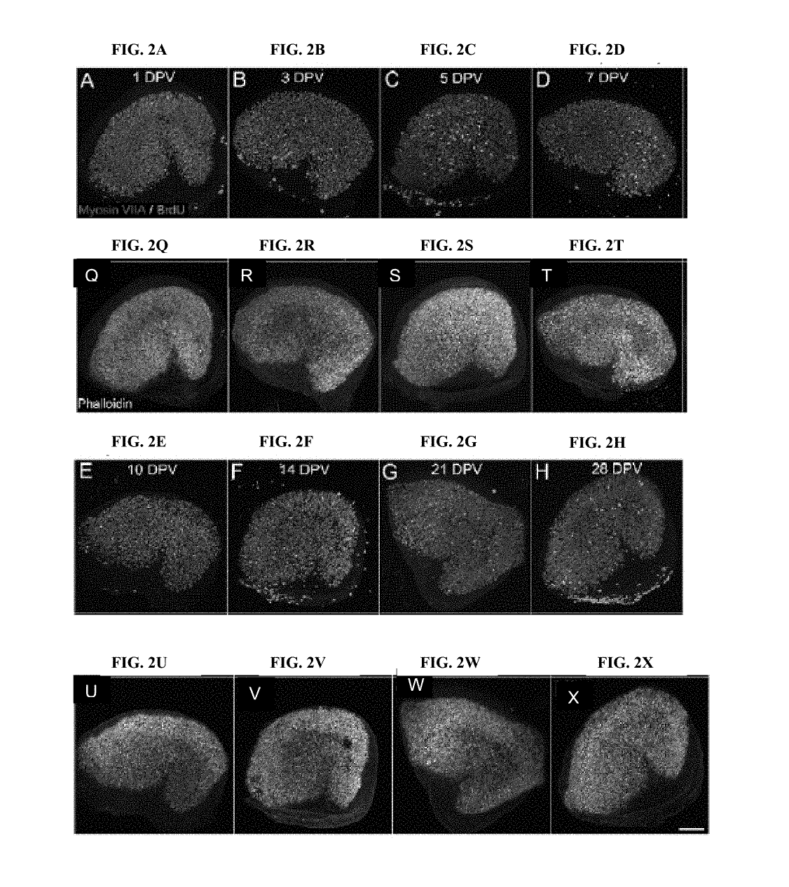

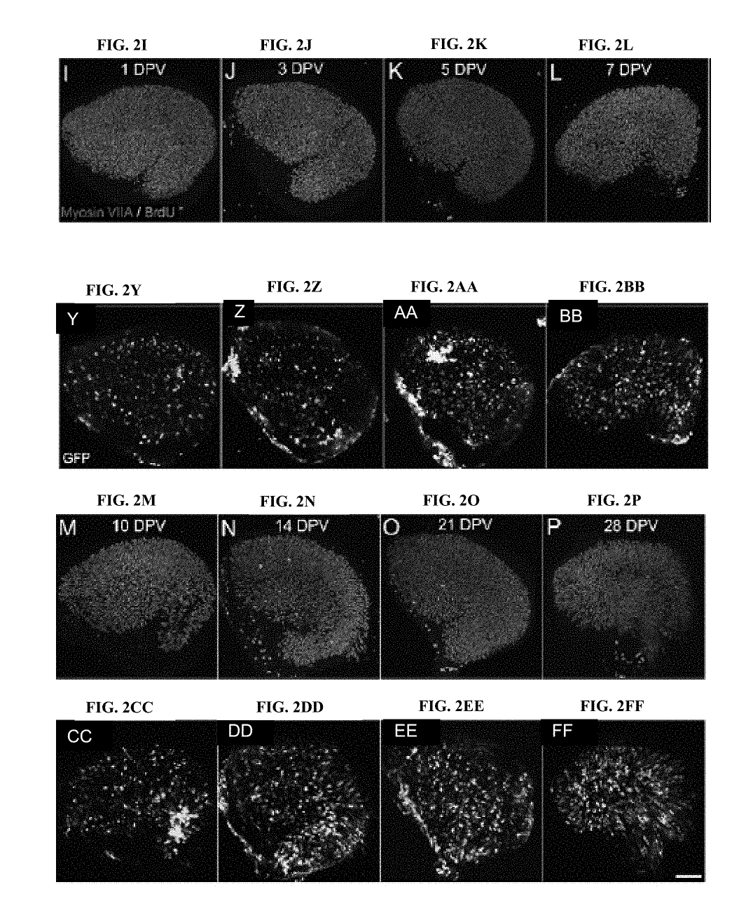

After characterizing the adenoviral transduction efficiency, it was determined whether ectopic expression of any of the four iPSC transcription factors could initiate cell cycle reentry of postmitotic supporting cells in vitro. For this, adult mouse utricles were co-infected with separate adenoviruses encoding mouse Oct3/4 (Ad.O, 5×107 TU/mL), mouse Klf4 (Ad.K, 2×108 TU/mL), mouse Sox2 (Ad. S, 5×107 TU/mL), and the T58A mutant of human c-Myc (Ad.MT58A, 2×108 TU/mL) under the control of CMV promoters and then BrdU was added to the culture medium for the remainder of the culture period to label any cells that entered S-phase (n=3-7 utricles per culture period). The T58A mutation confers resistance to degradation by preventing threonine phosphorylation (Chang et al., 2000, Mol. Cell Biol. 20:4309-4319; Gregory and Hann, 2000, Mol. Cell Biol. 20:2423-2435; Sears et al., 2000, Genes Dev 14:2501-2514; Salghetti et al., 1999, EMBO J 18:717-726). For controls, other utricles were infected with Ad.GFP alone (5×108 TU/mL; n=2-5 utricles per culture period). Twenty-four hours after washing out the virus, antibodies to BrdU did not label any nuclei in the sensory epithelium of utricles co-infected with Ad.O, Ad.K, Ad.S, and Ad.MT58A (FIGS. 2A, 2I). However, by 3 DPV, the co-infected utricles contained many BrdU-positive nuclei in their sensory epithelia (FIGS. 2B, 2J). BrdU-positive nuclei in the maculae at six later timepoints ranging from 5 to 28 DPV were also observed (FIGS. 2C-2H). Induction of S-phase entry appeared to be specific to utricles co-infected with the four iPSC transcription factors since comparatively minimal BrdU labeling was detected in the sensory epithelium of Ad.GFP-infected utricles (FIGS. 2I-2P).

Even at 28 DPV, the sensory epithelium maintained its integrity and mitten-like shape, and supporting cells and hair cells maintained compaction with tall aspect ratios (FIGS. 2Q-2X). Adenoviral vectors typically do not integrate into the host DNA, and they exhibit transient expression profiles in some cells, which may reduce the potential for tumorigenic effects from insertional mutagenesis or transformation (Stadtfeld et al., 2008, Science 322:945-949). Nevertheless, strong GFP expression persisted at qualitatively similar cellular densities for all 28 DPV (FIGS. 2Y-2FF). GFP expression also persisted in the surrounding non-sensory epithelium and underlying stromal tissue, suggesting that the replication-deficient adenovectors were not cleared or may have integrated into the host DNA. LacZ expression from adenoviral vectors has also been observed out to one month after delivery of adenovirus to mouse utricles in vivo (Kawamoto et al., 2001, Mol. Ther. 4:575-585).

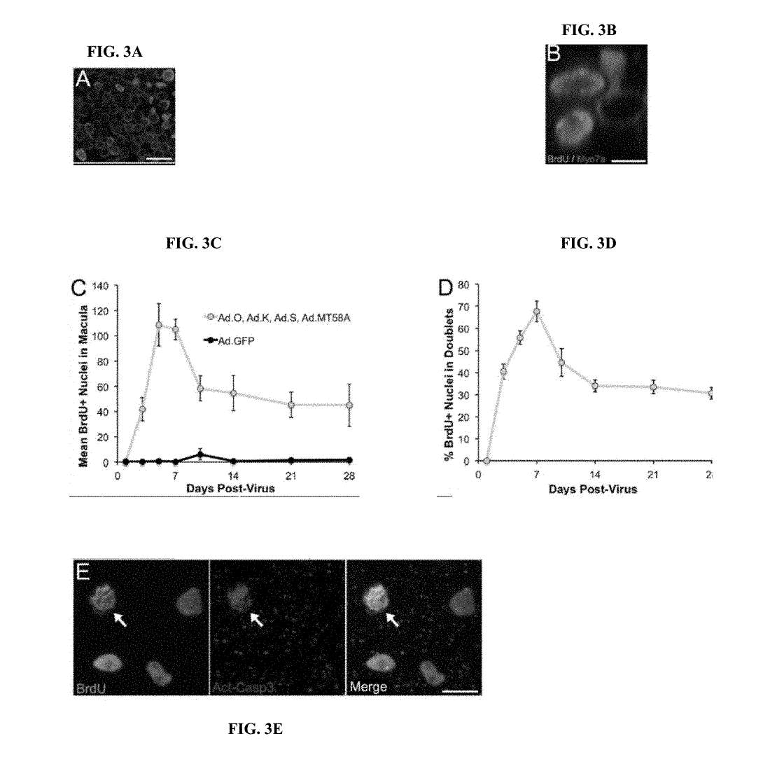

None of the BrdU-positive nuclei in the sensory epithelium labeled with antibodies to myosin VIIA, indicating that only supporting cells were entering S-phase and none of the labeled cells had differentiated into new hair cells when cultured with growth medium (FIGS. 3A-3B). Quantification of the mean number of BrdU-positive supporting cells revealed that S-phase entry peaked between 5 and 7 DPV (mean BrdU-positive nuclei at 7 DPV=105±8; FIG. 3C). The mean number of BrdU-labeled nuclei per sensory epithelium declined sharply by 45% from 7 to 10 DPV, and then decreased more gradually and eventually stabilized between 10 and 28 DPV (mean BrdU-positive nuclei at 28 DPV=45±17; FIG. 3C). Since BrdU was included in the culture medium throughout, a decline in the number of BrdU-positive nuclei indicates that the population of BrdU-labeled cells somehow became depleted, likely via cell death or exclusion from the sensory epithelium. Although not wishing to be bound to any particular theory, antibodies to activated caspase 3 co-labeled 2-3 BrdU-positive cells per sensory epithelium at 8 DPV (n=2 utricles), suggesting that the depletion of BrdU-positive cells was due to apoptosis (FIG. 3E). The small number of activated-caspase-3-labeled cells may have been due to rapid apoptosis (Loponen et al., 2011, PloS One 6:e27360).

Many of the BrdU-labeled nuclei were present in pairs, triplets, or quadruplicates, suggesting that some cells were completing mitosis and dividing (FIGS. 3A-3B, 3D). Quantification showed that the percentage of BrdU-positive nuclei per sensory epithelium appearing in doublets also peaked between 5 and 7 DPV (FIG. 3D; percentage of doublets at 7 DPV=68±8%). The percentage of doublets declined thereafter, following a similar trend as the mean number of BrdU-labeled nuclei (FIG. 3D; percentage of doublets at 28 DPV=31±3%). Although not wishing to be bound to any particular theory, together, the results suggest that many cells were passing through S-phase, completing M phase and cytokinesis, and then die. Since the levels of BrdU labeling eventually stabilized and some doublets were still detected at 28 DPV, a fraction of cells may be capable of surviving after induction of cell cycle reentry with iPSC transcription factors.

Of the Four iPSC Transcription Factors, c-MycT58A is Both Necessary and Sufficient for Inducing Reentry of Supporting Cells into the Cell Cycle

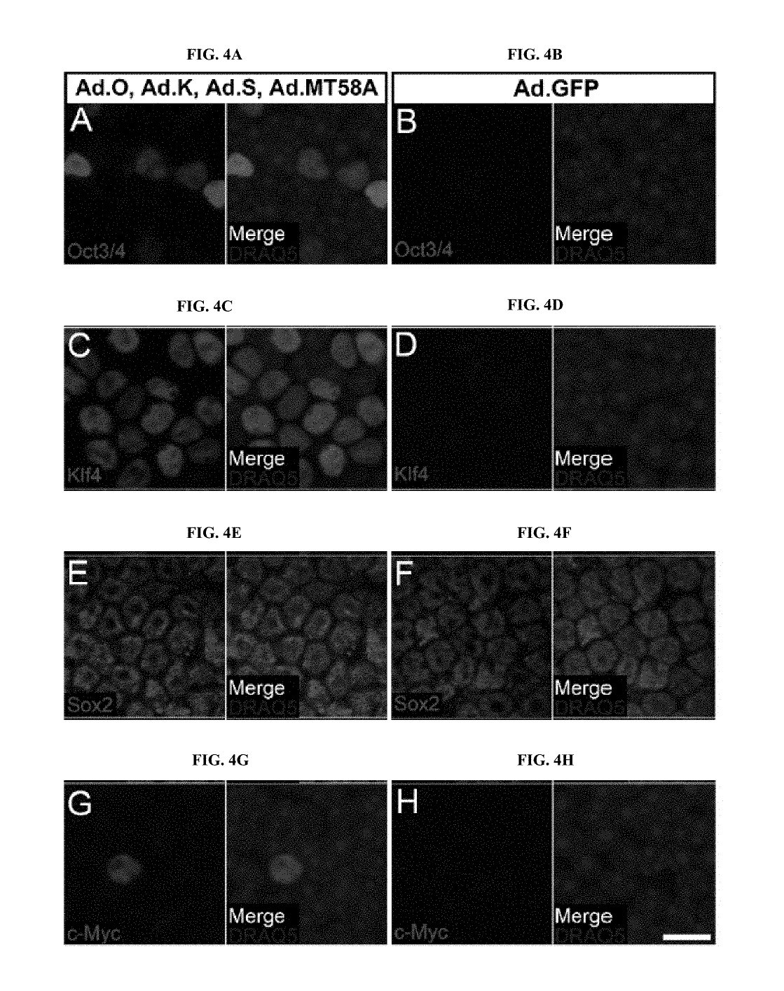

Antibody labeling of 3 DPV utricles that were separately infected with Ad.O, Ad.K, Ad. S, or Ad.MT58A (each at 5×10⁸ TU/mL) showed increased protein levels of Oct3/4, Klf4, and c-Myc in some supporting cell nuclei (FIGS. 4A, 4C, 4G). Oct3/4, Klf4, and c-Myc antibody labeling was not detectable in utricles infected with Ad.GFP, which indicated that the increased protein levels were a result of ectopic expression from Ad.O, Ad.K, and Ad.MT58A, respectively (FIGS. 4B, 4D, 4H). Sox2 is expressed in supporting cells and a subset of hair cells in the adult mouse utricle in vivo (Oesterle et al., 2008, J. Assoc. Res. Otolaryngol. 9:65-89), and the intensity of Sox2 antibody labeling did not appear to differ between Ad.S- and Ad.GFP-infected utricles (FIGS. 4E-4F).

Antibodies to Klf4 labeled the greatest number of nuclei, whereas c-Myc antibodies labeled<10 nuclei per sensory epithelium (FIGS. 4A, 4G). Ad.MT58A-infected utricles fixed and labeled with c-Myc antibodies at 1, 2, 5, and 10 DPV also contained similarly low numbers of c-Myc-positive supporting cells, as did antibody labeling for the hemagglutinin (HA) tag that was engineered into the c-MycT58A transgene.

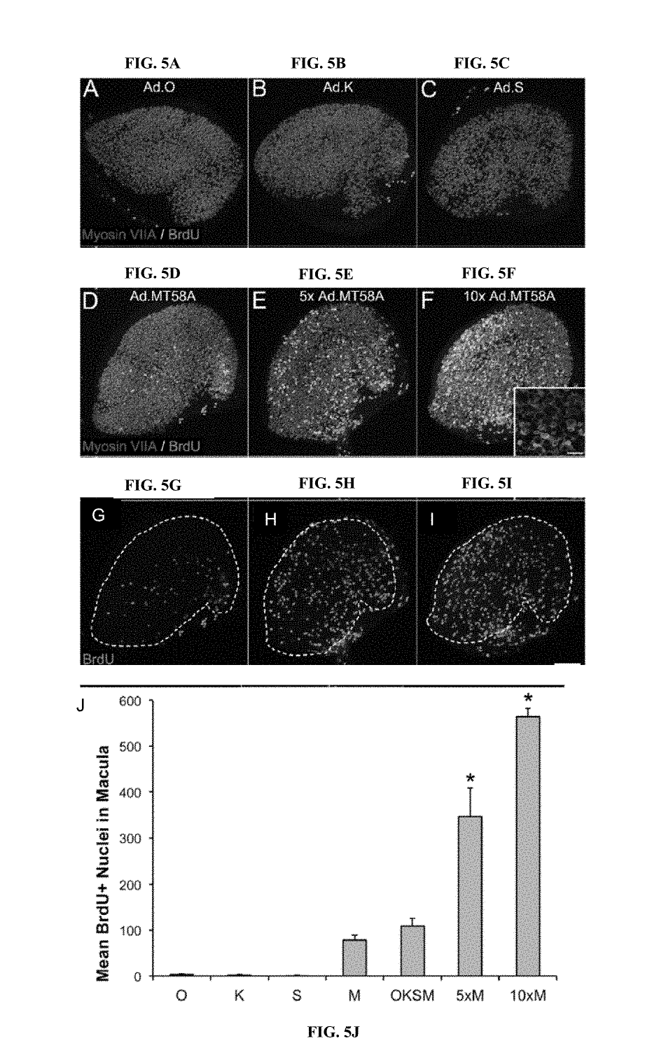

Since the ectopic protein levels appeared to differ for each factor, whether cell cycle reentry was dependent on one individual factor was determined by separately infecting adult mouse utricles with Ad.O (5×107 TU/mL), Ad.K (2×108 TU/mL), Ad.S (5×107 TU/mL), or Ad.MT58A (2×108 TU/mL) at viral concentrations identical to those used for the co-infection experiments. BrdU was included in the culture medium for the remainder after virus washout. Fixing the cultures at 5 DPV and labeling for BrdU revealed significant numbers of supporting cells that had reentered the cell cycle in utricles infected with Ad.MT58A compared to those infected with Ad.O, Ad.K, or Ad.S (FIGS. 5A-5D; mean BrdU-positive supporting cells in Ad.MT58A-infected utricles=79±10; p<0.05, One-way ANOVA with Tukey's Test of Multiple Comparisons; n=9 utricles). Increasing the concentration of Ad.MT58A 5- or 10-times significantly enhanced the numbers of BrdU-labeled nuclei at 5 DPV (FIGS. 5D-5J; mean BrdU-positive supporting cells at 1×109 TU/mL=3 57±65, mean BrdU-positive supporting cells at 2×109 TU/mL=564±19; p<0.05, One-way ANOVA with Tukey's Test of Multiple Comparisons; n=2-4 utricles), and increasing the concentration of Ad.O, Ad.K, or Ad.S to 1×10⁹ TU/mL did not result in significant S-phase entry. Thus, of the four transcription factors, c-MycT58A appears to be both necessary and sufficient for the induction of cell cycle reentry.

The levels of S-phase entry in Ad.MT58A-infected (2×108 TU/mL) utricles were similar to those in utricles co-infected with Ad.O (5×107 TU/mL), Ad.K (2×108 TU/mL), Ad.S (5×107 TU/mL), and Ad.MT58A (2×108 TU/mL; FIGS. 3C, 5J). When Ad.MT58A-infected utricles were double-labeled with antibodies to c-Myc and Ki-67, a protein that is upregulated during the active phases of the cell cycle (Kee et al., 2002, J. Neurosci. Methods 115:97-105), one nucleus in five specimens was detected that labeled with both antibodies, indicating that few actively cycling cells had detectable levels of c-MycT58A protein. While there were relatively few c-Myc-positive cells compared to the large number of proliferating cells, and although not wishing to be bound to any particular theory, it remains possible that autonomous expression of c-MycT58A in supporting cells induces cell cycle reentry, but c-MycT58A protein gets degraded to undetectable levels as the cells transition out of quiescence. Therefore, whether cell cycle reentry is driven by cell autonomous expression of c-MycT58A or whether ectopic expression in neighboring cells stimulates the proliferative response via paracrine signaling was not determined.

Supporting cells in Ad.MT58A-infected utricles can proceed to mitosis Many of the BrdU-positive nuclei in utricles infected with just Ad.MT58A appeared in doublets (FIGS. 5F, inset), and although not wishing to be bound to any particular theory, this suggests that these cells completed all phases of the cell cycle and divided. To further characterize the effects of Ad.MT58A infection on cell cycle progression in supporting cells, Ad.MT58A-infected utricles (1×109 TU/mL) fixed at 5, 7, and 10 DPV were co-labeled with antibodies to BrdU and Ki-67. Since BrdU is permanently incorporated into DNA during replication in S-phase, cells that label with antibodies to both Ki67 and BrdU have replicated their DNA and are still actively cycling. Cells that label for just BrdU have replicated their DNA and exited the cell cycle. Cells that label for just Ki67 have not yet replicated their DNA and are presumed to be in G1.

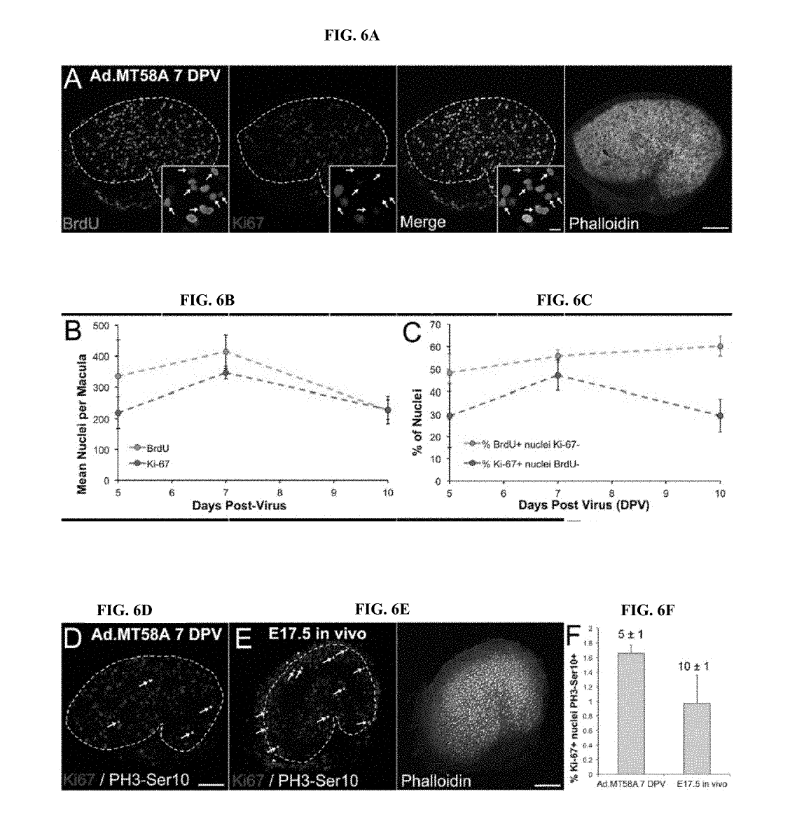

Similar to co-infected utricles, the mean number of BrdU-labeled nuclei per sensory epithelium in Ad.MT58A-infected utricles increased moderately from 5 to 7 DPV, and then declined by 47% between 7 and 10 DPV (FIGS. 3C, 6A-6B; mean BrdU-positive supporting cells at 7 DPV=414±54, mean BrdU-positive supporting cells at 10 DPV=228±44; n=4 utricles per culture period). The mean number of Ki-67-labeled nuclei per sensory epithelium exhibited a similar temporal pattern, but the percent decrease (34%) was smaller between 7 and 10 DPV (FIGS. 6A-6B).

Analysis of the percentage of Ki-67-positive supporting cells that did not label with antibodies to BrdU showed that cells in G1 accumulated from 5 to 7 DPV, and then many entered S-phase, exited the cell cycle, or died between 7 and 10 DPV (FIG. 6C; percentage of Ki-67-positive cells that were negative for BrdU at 7 DPV=47±7%; percentage of Ki-67-positive cells that were negative for BrdU at 10 DPV=30±7%). The percentage of the BrdU-positive population that did not label with antibodies to Ki-67 was also analyzed and steady increase was found from 48±8% at 5 DPV to 60±5% at 10 DPV, indicating cells progressively exited the cell cycle with increased time in culture (FIG. 6C). Regression analysis showed the increase was linear with an exit rate of 2.3% per day (r2=0.94).

The nuclei of cells that have just completed cytokinesis are typically smaller than nuclei in later phases of the cell cycle (Webster et al., 2009, J. Cell. Sci. 122:1477-1486), and the majority of BrdU-positive/Ki-67-negative nuclei appeared smaller than their BrdU-positive/Ki67-positive counterparts (arrows in FIG. 6A). To further assess whether most cycling cells were proceeding to mitosis, other cultures were fixed at 7 DPV and co-labeled with antibodies to phosphorylated serine 10 on histone H3 (PH3-Ser10) and Ki-67. Antibodies against PH3-Ser10 recognize chromatin condensation of mitotic cells in M phase (Hendzel et al., 1997, Chromosoma 106:348-360). Quantification showed the mean number of PH3-Ser10-labeled supporting cells and the percentage of the Ki-67-positive population that was also PH3-Ser10-positive were both low (FIGS. 6D, 6F; mean PH3-Ser10-positive nuclei per sensory epithelium=5±1, percentage of PH3-Ser10-positive/Ki-67-positive nuclei=1.7±0.4%; n=6 utricles). Preliminary analysis showed that the numbers of PH3-Ser10-positive nuclei were similar at 5, 6, and 8 DPV (n=2 utricles per culture duration, data not shown). Although not wishing to be bound to any particular theory, while these data do not rule out that progression to M phase may be inefficient in Ad.MT58A-infected utricles, they indicate that at least some supporting cells that reenter the cell cycle can proceed to mitosis.

The Percentage of Cells in M Phase is Similar in Ad.MT58A-Infected Utricles and Developing Utricles from Embryonic Mice

M phase is typically the most rapid phase of the cell cycle (Gordon and Lane, 1980, Cancer Research 40:4467-4472; Dover and Potten, 1988, J Cell Sci 89(Pt 3):359-364; Morgan DO, 2007, from The cell cycle: principles of control. Sunderland, Mass.: New Science Press), so immunocytochemisty with antibodies to PH3-Ser10 may detect few mitotic cells, particularly if a population of proliferating cells is not cell cycle synchronized. Thus, how the percentage of mitotic supporting cells in Ad.MT58A-infected utricles compared to those in the sensory epithelium of embryonic utricles developing in vivo was determined. For this, utricles were fixed from embryonic day 17.5 (E17.5) mice and labeled them with antibodies to Ki-67 and PH3-Ser10 to quantify the number of actively cycling cells in M phase within the sensory epithelium at the time of fixation. Terminal mitoses and expansion of the utricular sensory epithelium in mice do not cease until several days after birth, and significant numbers of cycling cells can still be detected at E17.5 (Burns et al., 2012, J. Neurosci. 32:6570-6577; Ruben, 1967, Acta Otolaryngol. (Suppl.) 220:1-44).

The mean number of PH3-Ser10-positive nuclei in the E17.5 sensory epithelium was significantly higher than in utricles infected with Ad.MT58A (FIGS. 6E-6F; mean PH3-Ser10-positive cells=10±1; p<0.05, Student's t-test; n=4 utricles). However, the percentage of the actively cycling population in M phase was comparable to that in Ad.MT58A-infected utricles at 7 DPV (FIGS. 6E-6F; percentage of PH3-Ser10-positive/Ki-67-positive nuclei in E17.5 utricles=1.0±0.1%; p>0.05, Student's t-test).

Although not wishing to be bound to any particular theory, the similar percentages suggest that Ad.MT58A infection may result in significant levels of cell division. A study examining cyclin D1 overexpression in adult mouse utricles found a similarly low percentage of PH3-Ser10-positive/Ki-67-positive supporting cells and concluded that M phase progression was rare and inefficient (Loponen et al., 2011, PloS One 6:e27360).

A Portion of the Supporting Cells that Reenter the Cell Cycle Remain Viable

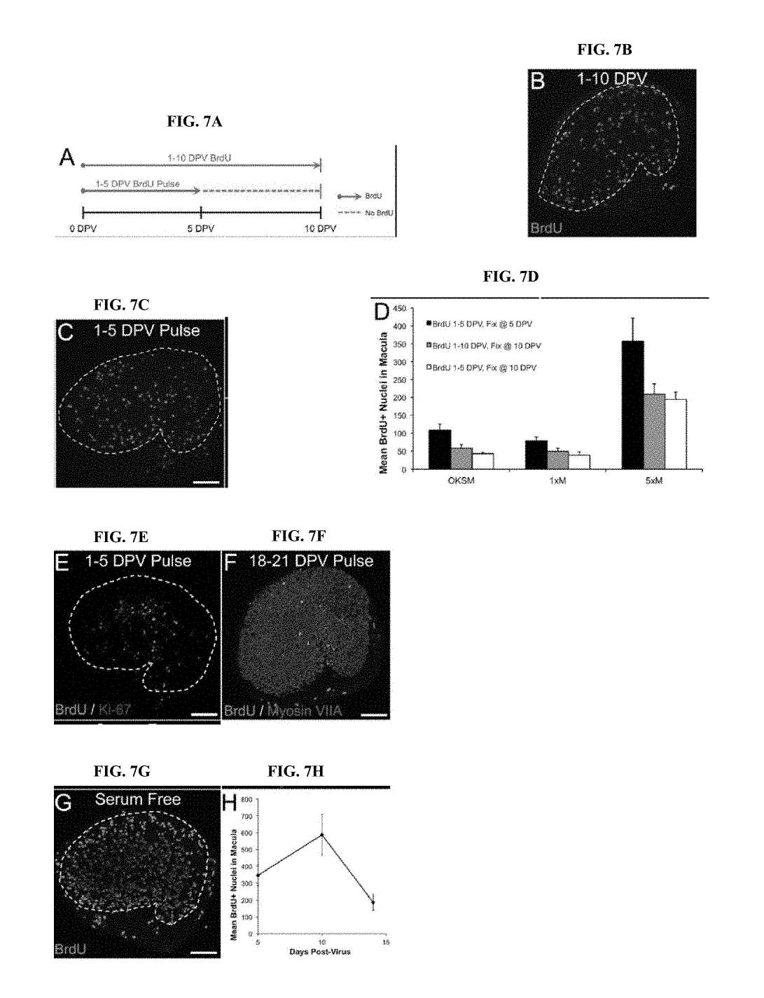

Knock-down of pocket proteins and cyclin dependent kinase inhibitors that limit cell cycle progression have been shown to stimulate division of hair cells and supporting cells; however, the progeny of these divisions rapidly die and can disrupt the integrity of the sensory epithelium (Lowenheim et al., 1999, Proc. Natl. Acad. Sci. U S A 96:4084-4088; Sage et al., 2005, Science 307:1114-1118; Sage et al., 2006, Proc. Natl. Acad. Sci. U S A 103:7345-7350; Yu et al., 2010, J. Neurosci. 30:5927-5936; Laine et al., 2007, J. Neurosci. 27:1434-1444; Mantela et al., 2005, Development 132:2377-2388; Chen et al., 2003, Nat. Cell. Biol. 5:422-426; Kanzaki et al., 2006, Hearing Research 214:28-36; Weber et al., 2008, Proc. Natl. Acad. Sci. U S A 105:781-785). Since many proliferating supporting cells underwent apoptosis after Ad.MT58A infection (FIGS. 3C, 6B), it was examined whether the BrdU-labeled cells detected at 10 DPV were cells that had just recently entered S-phase or whether they replicated their DNA early in the culture and then survived. When Ad.MT58A-infected utricles (1×10⁹ TU/mL) were cultured with BrdU for the first 5 DPV, washed out, and then cultured for 5 more days (i.e. to 10 DPV) in its absence, the mean number of BrdU-labeled nuclei per sensory epithelium was lower than in utricles subjected to continuous BrdU labeling for all 10 DPV (FIGS. 7A-7D; mean BrdU-positive nuclei per sensory epithelium at 10 DPV after 1-5 DPV BrdU pulse=194±21; n=4 utricles); however, this difference failed to reach significance (p>0.05, Student's t-test). Similar trends were observed when the same pulse-labeling regimen was used on cultures co-infected with Ad.O (5×107 TU/mL), Ad.K (2×108 TU/mL), Ad.S (5×107 TU/mL), and Ad.MT58A (2×108 TU/mL) or infected with the lower concentration of just Ad.MT58A (2×108 TU/mL; FIG. 7D).

The percent difference between the number of BrdU-labeled cells in utricles fixed at 5 DPV after continuous BrdU labeling and in utricles fixed at 10 DPV after the 1-5 DPV BrdU pulse was 44%, which indicates that 66% of cells entering S-phase prior to 5 DPV are able to survive out to 10 DPV. Furthermore, some of the surviving cells persisted for several weeks since BrdU-labeled nuclei were still detected in the sensory epithelium when pulse labeled with BrdU from 1-5 DPV and the cultures were not fixed until 21 DPV (FIG. 7E).

To determine whether supporting cells in Ad.MT58A-infected utricles retained the ability to enter S-phase after extended culture, the cultures were pulse labeled with BrdU from 18-21 DPV and fixed at 21 DPV. Labeling with antibodies to BrdU and Ki-67 revealed that supporting cells were still actively cycling and entering S-phase during this period (FIG. 7F). Although not wishing to be bound to any particular theory, together, the results suggest that a portion of the cells that reenter the cell cycle after Ad.MT58A infection remain viable, and some retain the ability to enter S-phase for at least 3 weeks in culture.

BrdU-Labeled Cells with Hair-Cell-Like Characteristics Appear after Culturing Ad.MT58A-Infected Utricles in Serum Free Medium

Exchanging growth medium for differentiation medium can promote the differentiation of otic progenitors into hair cells (Hu and Corwin, 2007, Proc. Nat. Acad. Sci. U S A 104:16675-16680; Oshima et al., 2010, Cell 141: 704-716; Montcouquiol and Kelley, 2003, J. Neurosci. 23:9469-9478). Differentiation medium is serum-free, however, and c-Myc-expressing cells can enter apoptosis after withdrawal of growth factors (Dang, 2012, Cell 149:22-35). Thus, it was surprising when an increase in the number of BrdU-labeled cells in Ad.MT58A-inected utricles (1×109 TU/mL) was observed after growth medium was exchanged for differentiation medium at 5 DPV and the cultures fixed at 10 DPV (FIG. 7G-7H; mean BrdU-positive nuclei per sensory epithelium at 10 DPV=588±121; n=4 utricles). Although not wishing to be bound to any particular theory, this could possibly be attributed to the T58A mutant, which reduces the sensitivity of c-MycT58A-expressing cells to apoptosis induced by serum deprivation (Sears et al., 2000, Genes Dev. 14:2501-2514; Gregory and Hann, 2000, Mol. Cell Biol. 20:2423-2435; Chang et al., 2000, Mol. Cell Biol. 20:4309-4319). The increase in the number of BrdU-labeled cells was only temporary, however, as the levels of labeled cells decreased 68% between 10 and 14 DPV (FIG. 7H; mean BrdU-positive nuclei per sensory epithelium at 14 DPV=186±47 BrdU-positive nuclei; n=4 utricles). The reasons for this temporary delay in cell death remain unclear, but serum deprivation lengthens G1 in murine fibroblasts overexpressing c-Myc, and although not wishing to be bound to any particular theory, switching to serum-free differentiation medium could have slowed cell cycle progression and the onset of apoptosis (Karn et al., 1989, Oncogene 4:773-787).

Since serum removal temporarily delayed cell death and led to an increase in the number of BrdU-labeled nuclei, it was examined whether the accumulation of cells that were potentially dividing would be accompanied by expansion of the sensory epithelium. Using myosin VIIA labeling to delineate the sensory epithelium, measurements were made of the macular area in Ad.MT58A- or Ad.GFP-infected utricles (1×109 TU/mL) cultured in differentiation medium from 5-10 DPV. Infection with Ad.MT58A led to very modest, but significant expansion of the sensory epithelium (mean macular area of Ad.MT58A-infected utricles=0.19±0.01 mm2, mean macular area of Ad.GFP-infected utricles=0.17±0.01 mm2; p=0.007, Student's t-test; n=11 utricles), which further supported the hypothesis that significant numbers of supporting cells in Ad.MT58A-infected utricles are able to progress to M phase and divide.

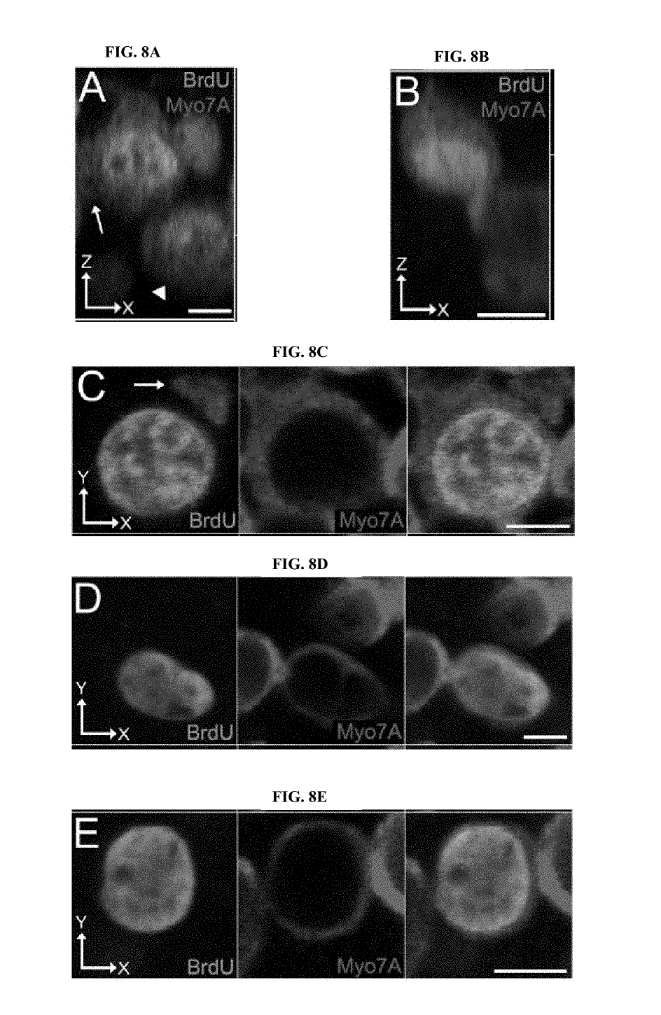

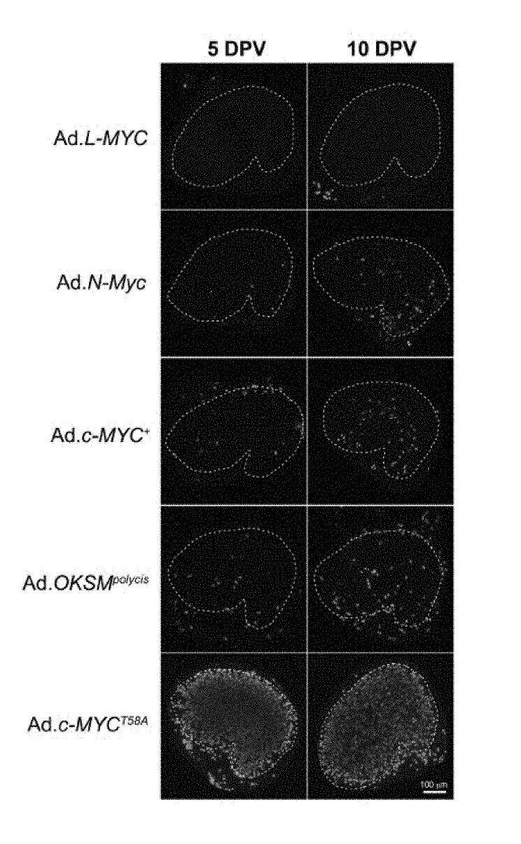

Although BrdU-labeled cells that also labeled with hair cell markers such as myosin VIIA in Ad.MT58A-infected utricles cultured with growth medium were never detected, Ad.MT58-infected utricles cultured with differentiation medium from 5-10 DPV contained a small number of BrdU-positive/myosin VIIA-positive cells (multiple examples shown in FIGS. 8A-8E; range of 1-4 BrdU-positive/myosin VIIA-positive cells per utricle; n=4 utricles). These cells had a chalice shape typical of hair cells, lacked connections with the basal lamina, and extended from the apical surface to the hair cell nuclear layer. Some were rounded and did not extend to the apical surface, suggesting they were damaged or dead (arrowhead in FIG. 8A). Most were paired with a BrdU-positive nucleus that did not label for myosin VIIA (arrows in FIGS. 8A, 8B), and although not wishing to be bound to any particular theory, this suggested a supporting cell had divided and one of the progeny had become a new hair cell. None displayed a prototypical, F-actin-rich hair bundle as determined with fluorescent phalloidin labeling, indicating these cells were not fully differentiated hair cells with functional mechanotransduction apparatuses. Although not wishing to be bound to any particular theory, Myosin VIIA is present in stereocilia, however, and small projections from the apical surface could be seen with myosin VIIA-labeling, suggesting that hair bundles may have been in the very early phases of formation (FIGS. 8A, 8B). Since a small number of GFP-expressing hair cells were observed after infection with Ad.GFP (FIGS. 1G-1H), and although not wishing to be bound to any particular theory, it is possible that the BrdU-positive/myosin VIIA-positive cells were preexisting hair cells that reentered the cell cycle after being transduced with Ad.MT58A. However, this appears unlikely since such cells were not observed in Ad.MT58-infected utricles cultured with just growth medium. Also, 1-3 BrdU-positive nuclei were observed in the Ad.GFP-infected control utricles that were cultured with differentiation medium from 5-10 DPV, but none of these cells were myosin VIIA-positive. Although not wishing to be bound to any particular theory, these results suggest that at least a small portion of supporting cells within intact vestibular organs may retain competency for differentiating into hair-cell-like cells after Ad.MT58A-induced cell cycle reentry.

Adenoviral Delivery of iPSC Transcription Factors

Described herein is an examination into how adenoviral delivery of the four iPSC transcription factors to adult mouse utricles affects regenerative potential within the sensory epithelium in vitro. It was observed that adenovectors encoding the T58A variant of c-Myc can initiate cell cycle reentry of postmitotic supporting cells. A portion of the cells that reenter the cell cycle survive for weeks in culture, proceed to mitosis, and appear to express the hair cell marker myosin VIIA under differentiating culture conditions. Cell cycle reentry also corresponded with a very modest, but significant expansion of the sensory epithelium, and although not wishing to be bound to any particular theory, this suggests that ectopic expression of MYC genes may be capable of stimulating regrowth of the sensory epithelium after cells have been lost to damage.

Suppression of MYC May Limit the Proliferative Potential of supporting cells in Adult Mammals

Although low numbers of supporting cells were observed ectopically expressing c-MycT58A, infection with Ad.MT58A led to robust supporting cell S-phase entry, and although not wishing to be bound to any particular theory, this suggests that c-Myc levels may be finely balanced within the utricle to suppress proliferation and maintain the postmitotic state. The half-life of c-MycT58A is shorter than that of Oct3/4 and Klf4 in some cell types (Klf4, 120 min in human esophageal cancer cells (Chen et al., 2005, Cancer Research 65:10394-10400; Tian et al., 2010, J. Biol. Chem. 285:7986-7994); Oct3/4, 90 min in mouse embryonic carcinoma cells (Saxe et al., 2009, PloS One 4:e4467); and c-MycT58A, 51-63 min in NIH3T3 and REF52 fibroblasts (Gregory and Hann, 2000, Mol. Cell Biol. 20:2423-2435; Sears et al., 2000, Genes Dev 14:2501-2514), but the differences in stability do not appear great enough to explain the large differences in the number of supporting cells expressing detectable levels of Oct3/4 and Klf4 compared to c-MycT58A (FIGS. 4A-4H). Although not wishing to be bound to any particular theory, this suggests that cells within the sensory epithelium of the adult mouse utricle may have active mechanisms for suppressing c-Myc protein levels, most likely at the post-transcriptional level since the CMV promoter efficiently drives gene expression in supporting cells (FIGS. 1A-1L and FIGS. 4A-4H).

Because there were few cells that labeled with antibodies to c-Myc and HA, it was not determined whether Ad.MT58A infection induced proliferation through autonomous or non-autonomous effects. Given c-Myc's well-documented role in directly regulating cell cycle machinery (Meyer and Penn, 2008, Nat. Rev. Cancer 8:976-990), it seems unlikely that supporting cell proliferation was stimulated by paracrine signaling from neighboring cells that were transduced by Ad.MT58A, especially since no c-Myc-labeled nuclei were detected in the sensory epithelium of Ad.GFP-infected controls. However, c-Myc overexpression has recently been shown to down-regulate the secretion of proteins that inhibit proliferation in a nontransformed epithelial cell line (Pocsfalvi et al., 2011, J Proteome Res 10:5326-5337).

Upstream signals that might limit c-Myc translation or promote its degradation in supporting cells have not been identified, but F-actin and the tumor suppressor E-cadherin accumulate at mammalian supporting-cell-supporting-cell junctions in coordination with the postnatal decline in regenerative capacity, and it has been posited that the signals and molecules responsible for junctional reinforcement may somehow limit regeneration (Collado et al., 2011, J. Neurosci. 31:11855-11866; Burns et al., 2008, J. Comp. Neurol. 511:396-414; Burns et al., 2012, J. Assoc. Res. Otalaryngol. 13:609-27; Burns et al., 2012, J. Neurosci. 32:6570-6577). In epithelial cells, c-Myc specifically activates or represses E-cadherin depending on the expression levels of the two proteins encoded by MYC, c-Mycl and c-Myc2, and increased E-cadherin expression in response to increased c-Myc levels can prevent cellular transformation (Gottardi et al., 2001, J Cell Biol 153:1049-1060; Batsche and Cremisi, 1999, Oncogene 18:5662-5671). Although not wishing to be bound to any particular theory, this sensitive feedback loop could potentially explain the concomitant decline in proliferation and accumulation of E-cadherin in mammalian supporting cells. c-Myc is a component of many signaling networks, having been estimated to regulate ˜30% of genes (Rahl et al., 2010, Cell 141:432-445), so potential repressors of c-Myc are numerous.

### MYC Gene Family Members are Potential Targets for Stimulating Cell Replacement in the Mammalian Inner Ear

Although not wishing to be bound to any particular theory, the supporting cell proliferation observed in adult mouse utricles after Ad.MT58A infection suggests that targeted upregulation of c-MycT58A or possibly even wild-type c-Myc may be a viable strategy for stimulating cell replacement in mammalian inner ear sensory epithelia. The MYC gene family members, which in mammals include the oncogenes MYC (c-Myc), MYCL (L-Myc), and MYCN (N-Myc), are basic Helix-Loop-Helix Leucine Zipper transcription factors that play a prominent role in regulating cell proliferation, growth, apoptosis, metabolism, and differentiation (Dang, 2012, Cell 149:22-35; Meyer and Penn, 2008, Nat. Rev. Cancer 8:976-990; Luscher and Vervoorts, 2012, Gene 494:145-160). Despite the robust proliferative response observed after Ad.MT58A infection, conditional deletion of c-Myc in the embryonic mouse inner ear has no phenotype, while deletion of N-Myc reduces proliferative growth and disturbs morphogenesis (Dominguez-Frutos et al., 2011, J. Neurosci. 31:7178-7189; Kopecky et al., 2011, Dev. Dyn. 240:1373-1390). Some proliferation was still detected in N-Myc mouse mutants, and it remains unclear whether this was due to compensation by another MYC family member like c-Myc. Although not wishing to be bound to any particular theory, these results suggest N-Myc could play a more prominent role in controlling proliferative regeneration in the mammalian inner ear, and overexpression of N-Myc in adults could be more efficient at inducing cell proliferation and promoting survival and differentiation into the correct cell types.

MYC can be a potent oncogene, so MYC gene therapy may not be a realistic approach for stimulating regeneration in humans (79 Yamanaka, 2009, Cell 137:13-170. In addition, c-MycT58A is more efficient than wild-type c-Myc at inducing immortalization and transformation (De Filippis et al., 2008, PloS One 3: e3310; Yeh et al., 2004, Nat. Cell Biol. 6:308-318), which increases the probability that some cells in these cultures were being immortalized or transformed. Although not wishing to be bound to any particular theory, cells were observed actively in the cell cycle and entering S-phase after 3 weeks in culture (FIGS. 7A-7H). Consequently, small molecules that target MYC and its binding partners may be more useful for a therapy that can be used in humans.

Similarities and Differences Between Proliferation Induced by Cyclin D1 and c-MycT58A

Adenovector-mediated expression of cyclin D1 (Ad.CD1) in cultured utricles from adult mice robustly induces supporting cell S-phase entry (Loponen et al., 2011, PloS One 6:e27360; Laine et al., 2010, Dev. Biol. 337:134-146). At 7 days after Ad.CD1 infection, 0.6% of the actively cycling cells (i.e. Ki-67-positive) were in M-phase (i.e PH3-Ser10-positive). This percentage was ˜4-times lower than in P9 mouse utricles infected with Ad.CD1, and it was ˜3-times lower than the percentage we observed in Ad.MT58A-infected utricles from adults (FIGS. 6A-6F). Although not wishing to be bound to any particular theory, a thorough analysis, which showed DNA damage in Ki-67-positive cells, minimal Aurora B kinase antibody labeling, few cells in cytokinesis, few cells completing multiple rounds of division, and no hyperplasia of the sensory epithelium led to the conclusion that ectopic cyclin D1 expression in supporting cells was inefficient at inducing cell cycle progression to mitosis.

Determining the exact percentage of cells capable of proceeding to M-phase may be difficult, especially if the proliferating population is not cell cycle synchronized. Similar numbers of PH3-Ser10-positive cells were detected at 5, 6, 7, and 8 DPV, and although not wishing to be bound to any particular theory, this suggests S-phase entry after Ad.MT58A infection is stochastic. Mitosis is typically the shortest phase of the cell cycle, making cells in M-phase the smallest fraction of the cycling population, and low numbers of mitotic cells were detected in both embryonic utricles from developing mice and Ad.MT58A-infected utricles from adult mice. While the percentage of cells in M phase was higher in embryonic and Ad.MT58A-infected utricles compared to Ad.CD1-infected utricles, the percentages in all cases were relatively low, underscoring the need for further characterization of cell cycle progression under all these conditions. It should be noted that many supporting cells are exiting the cell cycle around E17.5 in the utricle (Ruben, 1967, Acta Otolaryngol. (Suppl) 220:1-44), and in the rat retina, cell cycle length increases as more cells become postmitotic (Alexiades and Cepko, 1996, Dev Dyn 205:293-307). Although not wishing to be bound to any particular theory, it is possible that a significantly higher percentage of M phase cells may be present in the utricle at timepoints earlier than E17.5. In addition, ectopic c-Myc expression shortens the duration of G1 phase in murine fibroblasts and reduces the synchronization of cell cycle entry, consistent with our findings here (Karn et al., 1989, Oncogene 4:773-787). Although not wishing to be bound to any particular theory, the shortened G1 phase duration could increase the percentage of cells in M phase, potentially explaining why a higher percentage of M phase cells was found in utricles infected with Ad.MT58A compared to Ad.CD1.

Ectopic c-MycT58A Expression Used to Reprogram Supporting Cells into Multipotent Otic Progenitors

Although not wishing to be bound to any particular theory, the appearance of a small number of BrdU-positive/myosin-VIIA-positive cells after culturing in differentiation medium suggests that at least some supporting cells that reenter the cell cycle after Ad.MT58A infection retain otic identity and competency for differentiating into hair-cell-like cells. Although not wishing to be bound to any particular theory, the paucity of these cells could be attributed to the inability of the culture environment to replicate induction cues that may be present in vivo. Consistent with this notion, proliferation induced by hair cell death in newborn mouse utricles gave rise to new hair cells in vivo but not in vitro (Burns et al., 2012, J. Neurosci. 32:6570-6577). Alternatively, a small number of hair cells were observed that were transduced with Ad.GFP, and it is possible that the BrdU-positive/myosin VIIA-positive cells were pre-existing hair cells that reentered the cell cycle after being transduced with Ad.MT58A. Conditional deletion of the retinoblastoma protein or ectopic expression of the HPV-16 E7 oncogene within hair cells can drive their reentry into the cell cycle, so it is reasonable to suspect that ectopic expression of c-Myc may produce similar effects (Sage et al., 2005, Science 307:1114-1118; Sage et al., 2006, Proc. Nat. Acad. Sci. U S A 103:7345-7350; Yu et al., 2010, J. Neurosci. 30:5927-5936; Mantela et al., 2005, Development 132:2377-2388; Weber et al., 2008, Proc. Nat. Acad. Sci. U S A 105:781-785; Sulg et al., 2010, J Neurochem 112:1513-1526). Although not wishing to be bound to any particular theory, if the BrdU-positive/myosin VIIA-positive cells observed did originate from the supporting cell population infected with Ad.MT58A, then ectopic c-Myc expression may be capable of dedifferentiating supporting cells into cells with characteristics of otic progenitors.

The effects of Oct3/4, Klf4, and Sox2 are not observed until later in the iPSC reprogramming process (Ho et al., 2011, J. Cell. Physiol. 226:868-878), and these factors did not appear to influence proliferation over the time periods investigated. Expression of all four factors within individual supporting cells may have been unlikely since each was encoded by a separate adenovirus, and it remains to be determined whether simultaneous expression of Oct3/4, Klf4, and Sox2 would aid in directly reprogramming supporting cells into otic progenitors.

Transient expression of the four iPSC transcription factors under cardiac- or neural-promoting culture conditions directly reprograms fibroblasts into cardiomyocytes or neural stem/progenitor cells, respectively (Kim et al., 2011, Proc. Nat. Acad. Sci. USA. 108:7838-7843; Efe et al., 2011, Nat. Cell. Biol. 13:215-222). This short expression appears to induce epigenetic activation that results in an unstable, partially reprogrammed state that bypasses pluripotency but remains amenable to differentiation (Efe et al., 2011, Sci. Prog. 94:298-322). Similarly, it was hypothesize that when the four iPSC factors are delivered to terminally differentiated somatic cells in situ, the native organ environment may be able to suppress complete reprogramming to induced pluripotency while allowing direct reprogramming into lineage-restricted progenitor/stem cells. Lineage-specific transcription factors have been used to transdifferentiate cardiac fibroblasts into cardiomyoctes within intact mouse hearts in vivo, which demonstrates the feasibility of in situ reprogramming (Qian et al, 2012, Nature 485:593-598).

Furthermore, the bHLH transcription factor Atohl is both necessary and sufficient for hair cell differentiation during development, and ectopic expression of Atohl in situ appears to directly reprogram inner ear epithelial cells into hair cells in some instances (Bermingham et al., 1999, Science 284:1837-1841; Zheng and Gao, 2000, Nat. Neurosci. 3:580-586; Izumikawa et al., 2005, Nat. Med. 11:271-276; Shou et al., 2003, Mol. Cell. Neurosci. 23:169-179; Staecker et al., 2007, Otol. Neurotol. 28:223-231; Kelly et al., 2012, J. Neurosci. 32:6699-6710; Schlecker et al., 2011 Gene Ther. 18(9):884-90; Liu et al., 2012, J. Neurosci. 32:6600-6610). Directly reprogramming postmitotic supporting cells into hair cells could decrease the size of the supporting cell population without some form of nonautonomous cell replacement (Kelly et al., 2012, J. Neurosci. 32:6699-6710), which makes restoration of self-renewal capacity vital to the reprogramming strategy. Cyclins, cyclin dependent kinase inhibitors (CDKIs), and pocket proteins have been shown to regulate proliferation and cell cycle exit during development of the sensory epithelium, and a growing list of these genes have been targeted to force reentry of supporting cells into the cell cycle (Laine et al., 2010, Dev. Bio.1337:134-146; Lowenheim et al., 1999, Proc. Nat. Acad. Sci. U S A 96:4084-4088; Sage et al., 2005, Science 307:1114-1118; Sage et al., 2006, Proc. Nat. Acad. Sci. USA 103:7345-7350; Yu et al., 2010, J. Neurosci. 30:5927-5936; Laine et al., 2007, J. Neurosci. 27:1434-1444; Mantela et al., 2005, Development 132:2377-2388; Chen et al., 2003, Nat. Cell. Biol. 5:422-426; Kanzaki et al., 2006, Hearing Research 214:28-36; Weber et al., 2008, Proc. Nat. Acad. Sci. U S A 105:781-785; Sulg et al., 2010, J. Neurochem. 112:1513-1526; Oesterle et al., 2011, Cell Cycle 10:1237-1248; Chen and Segil, 1999, Development 126:1581-1590; Ono et al., 2009, Mol. Cell. Neurosci. 42:391-398; Rocha-Sanchez et al., 2011, J. Neurosci. 31:8883-8893; Huang et al., 2011, Cell Cycle 10:337-351). MYC controls the expression and activity of various cyclins, CDKIs, and pocket proteins (Dang, 2012, Cell 149:22-35; Luscher and Vervoorts, 2012, Gene 494:145-160), which may make it more suitable for orchestrating the complex fluctuations of cell cycle proteins that are necessary to drive efficient progression through the various restriction points. Combining forced cell cycle reentry—either via a MYC family member or direct targeting of cell cycle machinery—with ectopic Atohl expression is an alternative approach. Overall, ectopic MYC expression has been found to show promise for restoring proliferative capacity to inner ear sensory epithelia and may be used to stimulate regeneration.

### Example 2

### Myc Family Members

Experiments were conducted to determine whether other Myc family members or wild-type (WT) c-Myc can stimulate S-phase entry of adult mammalian supporting cells.

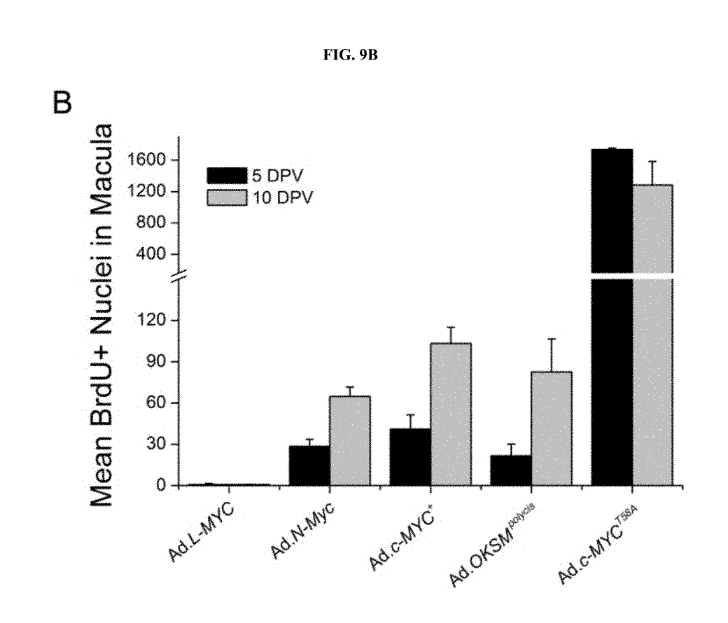

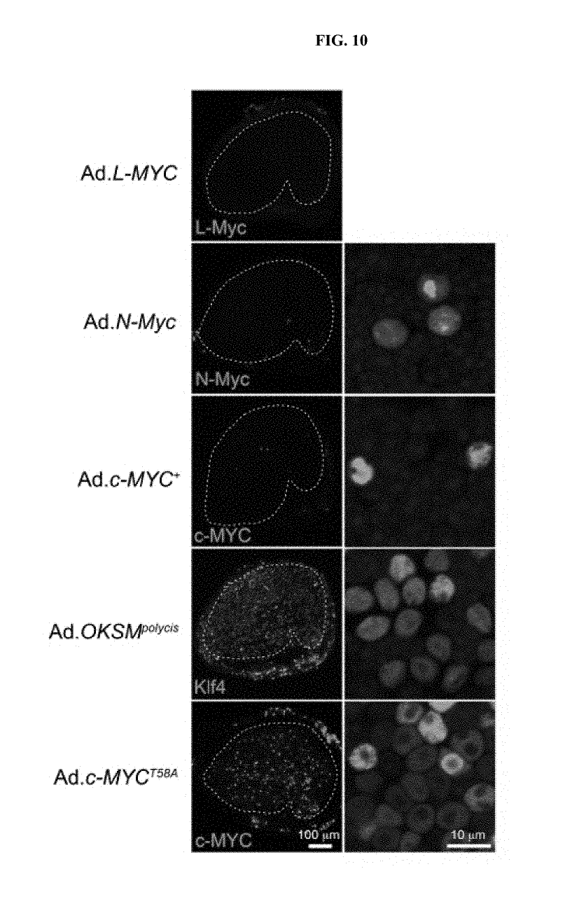

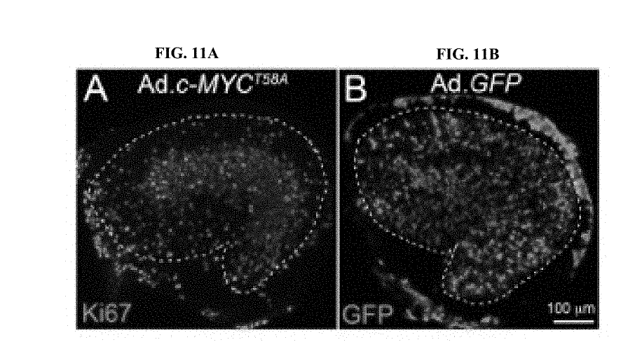

Briefly, cultured adult mouse utricles were infected with adenoviruses that express human WT L-Myc, mouse WT N-Myc, or human WT c-Myc under control of a CMV promoter (1×10¹⁰ TU/mL). Labeling with BrdU for 5 DPV showed that N-Myc and c-Myc induced significant S-phase entry of supporting cells within the sensory epithelium, whereas few to no BrdU-positive nuclei were detected in the sensory epithelium of utricles infected with Ad.L-Myc (FIGS. 9A-9B). Immunohistochemistry revealed that infection with Ad.N-Myc and Ad.c-Myc (each at 1×10¹⁰ TU/mL) led to increased levels of N-Myc and c-Myc protein in some supporting cells (FIG. 10).

Extension of the BrdU labeling protocol to 10 DPV revealed a moderate increase in the number of BrdU-postive supporting cells in utricles infected with Ad.N-Myc and Ad.c-Myc (FIGS. 9A-9B). This result contrasted with the decline in BrdU-positive nuclei observed between 5 and 10 DPV in utricles infected with Ad.c-MycT58A, although the magnitude of S-phase entry was significantly higher at both 5 and 10 DPV in utricles infected with Ad.c-MycT58A at a similar viral concentration (FIGS. 9A-9B).

To determine whether co-expression of all four iPS factors within individual supporting cells alters the proliferative phenotype induced by c-Myc, cultured adult mouse utricles were infected with adenoviruses containing a polycistronic vector that encodes mouse WT Oct3/4, mouse WT Klf4, mouse WT Sox2, and mouse WT c-Myc under control of the same CMV promoter (Ad.OKSMpolycis; 1×10¹⁰ TU/mL). BrdU labeling through 5 and 10 DPV showed a similar pattern of S-phase entry as in utricles infected with Ad.c-Myc (FIGS. 9A-9B).

Without wishing to be bound by any particular theory, it is believed that the T58A mutation on c-Myc enhances S-phase entry while promoting cell death. Since the T58A mutation confers stability on the c-Myc protein, the pro-death effects of Ad.c-MycT58A could be due to accumulation of c-Myc in postmitotic cells that would otherwise be capable of suppressing ectopic c-Myc expression. This is supported by experiments showing that at sufficiently high concentrations of Ad.c-MycT58A (1×10¹⁰ TU/mL), elevated levels of ectopic c-Myc protein can be detected in many more supporting cells than previously appreciated, which suggests that extreme levels of ectopic expression must be achieved to detect accumulated c-Myc protein in most supporting cells (FIG. 10). Such cells may be in a configuration that is not suitable for cell cycle reentry, and in response, they may initiate apoptosis after progressing into M phase. Under this scenario, overexpression of c-Myc or N-Myc does not result in significantly elevated levels of protein in these cells due to the shorter protein half-life compared to c-MycT58A. Cyclin D1 may have a longer half-life and may therefore be more efficient at driving cell cycle reentry than Myc genes; however, it may be insufficient to drive progression into M phase after cell cycle reentry (Loponen et al., 2011), which would arrest most cells before triggering apoptosis in M. Without wishing to be bound by any particular theory, this could explain the high numbers of BrdU-positive nuclei that accumulate in the sensory epithelium of utricles infected with adenoviruses that encode cyclin D1 (Loponen et al., 2011).

Moreover, a small subset of cells may be in a configuration that is more amenable to proliferation (FIGS. 11A-11B). Accumulation of Myc proteins may not pose a risk to this subset, and they may have lower Myc suppression activity. Overexpression of c-MycT58A, c-Myc, or N-Myc in these cells leads to protein accumulation, cell cycle reentry, and progression through M phase without apoptosis. Without wishing to be bound by any particular theory, it is believed that identifying and targeting this population for multiple rounds of proliferation may be a desirable strategy for stimulating cell replacement in the inner ear. Therefore, one aspect of the invention included targeting a subpopulation of supporting cells that may be more amenable to cell cycle progression.

N-Myc, c-Myc, c-MycT58A, and cyclin D1 can all stimulate aberrant S-phase entry of postmitotic supporting cells, while L-Myc cannot. Adding the other three iPS factors does not augment the proliferative profile, at least for the first 10 DPV. c-MycT58A induces more robust S-phase entry than c-Myc, N-Myc, or OKSMpolycis, but the T58A mutation promotes more cell death, at least by 10 DPV. M phase progression to cytokinesis is stochastic in all instances, and does not depend on the transgene or cell type. Therefore, the invention should not be limited to only c-MycT58A. That is, the invention includes the use of cyclin D1, or other upstream or downstream targets in the c-Myc signaling pathway.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-12. (canceled)

13. A method of inducing post-mitotic inner ear supporting cells to expand and differentiate into a population of inner ear hair cells, the method comprising administering to a population of post-mitotic inner ear supporting cells an agent that increases c-myc expression in the post-mitotic inner ear supporting cells, whereby cells in the population of post-mitotic inner ear supporting cells enter the cell cycle and expand to form progeny cells that differentiate into a population of inner ear hair cells.

14. The method of claim 13, wherein the agent is an activator of protein kinase C.

15. The method of claim 14, wherein the agent is a phorbol ester analogue, a bombesin analogue, forskolin, or sulindac.

16. The method of claim 13, wherein after a period of time where c-MycT58A protein is expressed in the post-mitotic inner ear supporting cell, the expression or function of c-MycT58A protein is inhibited by contacting the mitotic inner ear supporting cell with an inhibitor.

17. The method of claim 16, wherein the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

18. A method of inducing post-mitotic inner ear supporting cells to expand and differentiate into a population of inner ear hair cells, the method comprising transducing a population of post-mitotic inner ear supporting cells with a vector encoding c-MycT58A protein, whereby cells in the population of post-mitotic inner ear supporting cells enter the cell cycle and expand to form progeny cells that differentiate into a population of inner ear hair cells.

19. The method of claim 18, wherein the vector is a plasmid or a viral vector.

20. The method of claim 19, wherein the viral vector is a retroviral vector, a lentiviral vector, an adenovirus vector, or an adenovirus-associated vector (AAV).

21. The method of claim 18, wherein after a period of time where c-MycT58A protein is expressed in the post-mitotic inner ear supporting cell, the expression or function of c-MycT58A protein is inhibited by contacting the mitotic inner ear supporting cell with an inhibitor.

22. The method of claim 21, wherein the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.
